(19) United States
(12) Patent Application Publication
(10) Pub. No.: US 2009/0317456 A1
(43) Pub. Date: Dec. 24, 2009

(54) USE OF ONCOLYTIC VIRUSES AND ANTIANGIOGENIC AGENTS IN THE TREATMENT OF CANCER

(75) Inventors: Matthias Karrasch, Erlangen (DE); Axel Mescheder, Woerthsee (DE)

Correspondence Address: CLARK & ELBING LLP, 101 FEDERAL STREET, BOSTON, MA, 02110, US

(73) Assignee: MediGene AG, Planegg/Martinsried (DE)

(21) Appl. No.: 12/445,019

(22) PCT Filed: Oct. 15, 2007
(86) PCT No.: PCT/EP07/08930

§ 371 Date: Jul. 22, 2009

Related U.S. Application Data
(60) Provisional application No. 60/851,598, filed on Oct. 13, 2006.

(51) Int. Cl. A61K 35/76; A61P 35/00; A61K 39/395; A61K 9/127

(52) U.S. Cl. 424/450; 424/93.6; 424/93.2; 424/133.1

(57) ABSTRACT

The present invention relates to a combination of at least one oncolytic virus and at least one antiangiogenic agent and to the use of this combination in tumor therapy.

The present application claims the priority of U.S. 60/851,598, herewith incorporated by reference.

The present invention relates to the combined use of at least one oncolytic virus and at least one antiangiogenic agent in tumor treatment.

Malignant tumors become more and more common and they pose a significant threat to human lives. There are conventional means to treat malignant tumors, such as surgery, chemotherapy and radiotherapy. The type or stage of the cancer can determine which of the three general types of treatment will be used. An aggressive, combined modality treatment plan can also be chosen e.g. surgery can be used to remove the primary tumor and the remaining cells are treated with radiation therapy or chemotherapy (Rosenberg, 1985). In general, chemotherapeutic agents and radiotherapy are unable to distinguish cancer cells from normal cells. Moreover, these therapies are inefficient for patients suffering from tumors in an advanced stage, therefore people tried to develop new strategies. Although there were great expectations in tumor gene therapy, there has been no clinical breakthrough so far (Liu, 2005). The use of hormone therapy (Cersosimo and Carr, 1996) and immunotherapy (Matzku and Zoller, 2001) remains limited to distinct cases and cancer types. Research to identify more effective drugs for treating advanced disease continues.

The use of replication-competent viral vectors, such as herpes simplex virus type 1 (HSV-1) vectors, have attracted much interest for the specific killing of tumor cells and this oncolytic virotherapy is being evaluated in clinical trials (Post, 2004), because such viruses can replicate and spread in situ, exhibiting oncolytic activity through direct cytopathic effect (Kirn, 2000,) thus overcoming the delivery problems of gene therapy. A number of oncolytic HSV-1 vectors have been developed that have mutations in genes associated with neurovirulence and/or viral DNA synthesis, in order to restrict replication of these vectors to transformed cells and not cause disease (Martuza, 2000). Since these viruses kill cells by oncolytic mechanisms differing from standard anticancer therapies, their use in combination with chemo-, radio-, and gene therapies have been examined (Post, 2004).

One rationale for using oncolytic viruses is that viral replication in infected tumor cells permits in situ viral multiplication and spread of viral infection throughout the tumor mass. Improved understanding of the life cycle of viruses has evidenced multiple interactions between viral and cellular gene products, which have evolved to maximize the ability of viruses to infect and multiply within cells. Other modes of action that may play a role are the induction of apoptosis (Coukos et al., 2000) and the induction of an immune response against the virally infected host cell that generates an anti-tumor response through the activation of the cellular immune system (Varghese et al., 2006). Differences in viral-cell interactions between normal and tumor cells have emerged that have led to the design of a number of genetically engineered viral vectors that selectively kill tumor cells while sparing normal cells.

The field of cancer research has seen a marked shift in the past decade towards the exploration and development of non-conventional antitumor agents.

One of the most widely studied approaches to therapy during this period has been that of antiangiogenesis (Isayeva et al., 2004). There is substantial preclinical and clinical evidence that angiogenesis plays a role in the development of tumors and the progression of malignancies. Inhibiting angiogenesis has been shown to suppress tumor growth and metastasis in many preclinical models. These benefits have translated to the clinic with both marketed and investigational antiangiogenetic agents (Lenz, 2005). Tumors require nutrients and oxygen in order to grow, and new blood vessels, formed by the process of angiogenesis, provide these substrates. The key mediator of angiogenesis is vascular endothelial growth factor (VEGF) which is induced by many characteristics of tumors, most importantly hypoxia. Therefore, VEGF and its receptors are the most prominent targets of antiangiogenic compounds in anticancer therapies. In addition, VEGF is easy to access as it circulates in the blood and acts directly on endothelial cells. VEGF-mediated angiogenesis is rare in adult humans (except wound healing and female reproductive cycling), and so targeting the molecule should not affect other physiological processes (Ferrara, 2005). The published clinical trials and subsequent FDA approval (in February 2004) of the anti VEGF monoclonal antibody Bevacizumab (Avastine®, Genentech) for the treatment of colorectal cancer marked a milestone for antiangiogenesis therapy (Wakelee and Schiller, 2005).

In addition to a number of agents targeting the VEGF pathway, several other factors are of interest as target for antiangiogenic compounds as well. These include integrins, matrix metalloproteinases (MMPs), protein kinase C beta (PKCβ), and endogenous antiangiogenic factors. Moreover, cartilage is a natural source of material with strong antiangiogenic activity. Purified antiangiogenic factors from shark cartilage such as Neovastat, U-995 and Squalamine already showed strong antitumor activity (Cho and Kim, 2002). Unlike these antiangiogenic drugs that inhibit the formation of new vessels, vascular targeting agents (VTAs) occlude the pre-existing blood vessels of tumors thereby causing tumor cell death (Thorpe, 2004). Furthermore, Thalidomide or one of its immunomodulatory analogs have been implicated for anticancer therapy among other numerous effects on the body's immune system due to their antiangiogenic activity (Teo, 2005).

Many receptors have been selected as viable drug discovery targets. One particular class of receptors that have received much interest and so far relatively good success are the receptor protein tyrosine kinases. Typically, receptor tyrosine kinases are activated following the binding of the peptide growth factor ligand to its receptor. The receptor tyrosine kinases play crucial roles in signal transduction pathways that regulate a number of cellular functions, such as cell differentiation and proliferation, both under normal physiological conditions as well as in a variety of pathological disorders. A variety of different tumor types have been shown to have dysfunctional receptor tyrosine kinases. Irrespective of the cause, this leads to the over-activity of the particular receptor tyrosine kinase system and in turn to the aberrant and inappropriate cellular signalling within the tumor cell.

The EGF receptor, PDGF receptor, FGF receptor and VEGF receptor have been selected as molecular targets for drug discovery programmes, with the main emphasis of interest being on their role in oncology. Most recently known tyrosine kinase inhibitors, target more than one of these receptors especially when tested in higher concentration (Cardones, 2006). Since these receptors act alone and in concert on multiple steps resulting in changes in cell proliferation, permeability and migration and at the bottom line on tumor growth and blood vessel formation inhibitors targeting more than one of these tyrosine kinases are often most effective e.g. in the treatment of tumor diseases.

Furthermore, for some tyrosine kinase receptors it was shown that they upon ligand binding homo- and heterodimerize with other family molecules and for the tyrosine kinase domain of each molecule to transphosphorylate its partner: thus EGFR (also known as ErbB1) can mediate the activation of itself as well as ErbB2-4 (Grant, 2002).

Cationic liposomes can be used to selectively deliver agents to angiogenic endothelial cells. This method involves injecting, preferably systemically into the circulatory system and more preferably intravenously, cationic liposomes which comprise cationic lipids and a compound which inhibits angiogenesis and/or includes a detectable label (Strieth et al., 2004). After administration, the cationic liposomes selectively associate with angiogenic endothelial cells meaning that they associate with angiogenic endothelial cells at a five fold or greater ratio (preferably ten fold or greater) than they associate with corresponding, quiescent endothelial cells not undergoing angiogenesis. When the liposomes associate with angiogenic endothelial cells, they are taken up by the endothelial cell. This preferential uptake raises the possibility of using cationic liposomes to target diagnostic or therapeutic agents selectively to angiogenic blood vessels in tumors (Thurston et al., 1998).

Although surgery, chemotherapy and radiotherapy remain the standard approaches for cancer patients, a plateau has been reached in their efficacy. Their success rate remains limited, primarily due to limited accessibility of the tumor tissue, their toxicity and resulting side effects especially on non-cancer cells, development of multi-drug resistance and the dynamic heterogeneous biology of the growing tumors.

Beyond the primary tumor, metastasis is the most common cause of death in cancer patients with angiogenesis being one of the most important factors (Wittekind and Neid, 2005). Moreover, the results of a large body of preclinical studies and clinical trials suggest that targeting VEGF, integrins, MMPs, PKCβ and other factors by antiangiogenic compounds represents a significant contribution to cancer therapy. Moreover, promising antitumor activity due to antiangiogenic properties could have been shown in the past for drugs purified from shark cartilage, VTAs, Thalidomide and some of its immunomodulatory analogs. In addition, compound loaded cationic liposomes preferentially taken up by angiogenic endothelial cells can e.g. destroy the endothelial cell, inhibit further angiogenesis and/or tag the endothelial cell so that it can be detected by an appropriate means.

In a first aspect, the present invention relates inter alia to a combination of at least one oncolytic virus and at least one antiangiogenic agent.

In the context of the present invention, it has been found that cetuximab (Erbitux®), a EGFR tyrosine kinase inhibitor and antiangiogenic agent, has beneficial effects when administered in combination with HSV, an oncolytic virus.

Therefore, in accordance with the present invention, it is assumed that applying a combination therapy comprising at least one oncolytic virus and at least one antiangiogenic agent in particular in patients suffering from tumorigenic diseases potentiates their effects compared to each treatment modality alone.

This treatment can be used in advanced tumor disease, e.g. second or third line treatment, or in first line treatment.

Prior to describing the invention in further detail, the terms used in this application are defined as follows unless otherwise indicated.

As used herein, the transitional term “comprising” is open-ended. A claim utilizing this term can contain elements in addition to those recited in such claim. Thus, for example, the claims can read on treatment regimens that also include other therapeutic agents or therapeutic virus doses not specifically recited therein, as long as the recited elements or their equivalent are present.

The terms “treatment”, “treating”, “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease.

“Treatment” as used herein covers any treatment of a disease in a mammal, particularly a human, and includes:
(a) preventing the disease or symptom from occurring in a subject which may be predisposed to the disease or symptom but has not yet been diagnosed as having it;
(b) inhibiting the disease symptom, i.e., arresting its development; or
(c) relieving the disease symptom, i.e., causing regression of the disease or symptom.

The term “angiogenesis” refers to a process of tissue vascularization that involves the development of new vessels. Angiogenesis may occur via one of three mechanisms (Blood and Zettler, 1990):
(1) neovascularization, where endothelial cells migrate out of pre-existing vessels beginning the formation of the new vessels;
(2) vasculogenesis, where the vessels arise from precursor cells de novo; or
(3) vascular expansion, where existing small vessels enlarge in diameter to form larger vessels.

As used herein, “tumor cell formation and growth” describes the formation and proliferation of cells that have lost the ability to control cellular division, thus forming cancerous cells.

As indicated, the viruses selectively kill neoplastic cells including malignant and benign neoplastic cells.

As used herein, “neoplastic cells” or “neoplasia” refers to abnormal, disorganized growth in a tissue or organ, usually forming a distinct mass. Such a growth is called a neoplasm, also known as a tumor.

For purposes of the invention, neoplastic cells include cells of tumors, neoplasms, carcinomas, sarcomas, leukemias, lymphomas, and the like. Any virus capable of replication selectively in neoplastic cells may be utilized in the invention.

As used herein, “potentiate” means additive or even synergistic increase of the level of cell killing above that seen for one treatment modality alone.

The term “combined amount effective to kill the cell” means that the amount of the antiangiogenic compound and virus are sufficient so that, when combined within the cell, cell death is induced. The combined effective amount of the agents will preferably be an amount that induces more cell death than the use of either element alone.

According to the invention, the term “inhibitor” means either that the given compound is capable of inhibiting the activity of the respective protein or other substance in the cell at least to a certain amount. This can be achieved either by a direct interaction of the compound with the given protein or substance (“direct inhibition”) or by an interaction of the compound with other proteins or other substances in or outside the cell which leads to an at least partial inhibition of the activity of the protein or substance (“indirect inhibition”).

As a suitable assay for measuring in vitro angiogenesis is the ECM625 assay kit by CHEMICON, Temecula, Calif. The CHEMICON In Vitro Angiogenesis Assay Kit provides a convenient system for evaluation of tube formation by endothelial cells in a convenient 96-well format. When cultured on ECMatriX™, a solid gel of basement proteins prepared from the Engelbreth Holm-Swarm (EHS) mouse tumor, these endothelial cells rapidly align and form hollow tube-like structures. Tube formation is a multi-step process involving cell adhesion, migration, differentiation and growth. ECMatrix™ consists of laminin, collagen type IV, heparan sulfate proteoglycans, entactin and nidogen. It also contains various growth factors (TGF-beta, FGF) and proteolytic enzymes (plasminogen, tPA, MMPs) that occur normally in EHS tumors. It is optimized for maximal tube-formation. The CHEMICON In Vitro Angiogenesis Assay Kit represents a simple model of angiogenesis in which the induction or inhibition of tube formation by exogenous signals can be easily monitored. For assaying inhibitors or stimulators of tube formation, simply premix the endothelial cell suspension with different concentrations of the inhibitor or stimulator to be tested, before adding the cells to the top of the ECMatrix™. The assay can be used to monitor the extent of tube assembly in various endothelial cells, e.g. human umbilical vein cells (HUVEC) or bovine capillary endothelial (BCE) cells. For references see data sheet/insert of CHEMICON for ECM625, April 2002, Revision B: 41075 and Nam J O et al. (2003).

Similarly, the term “effective amount” is an amount of an antiangiogenic agents and a virus that, when administered to a mammal in combination, is effective to kill cells in the mammal. this is particularly evidenced by the killing of cancer cells within an animal or human subject that has a tumor. The methods of the instant invention are thus applicable to a wide variety of animals, including mice and hamsters.

As a suitable assay for measuring in vivo angiogenesis the Cultrex® DIVAA™ Angiogenesis Assay Kit, Tevigen Inc. Gaithersburg Md., is suitable (DIVAA Cultrex Instructions for Use (2004), MDGuedez L et al. (2003). The Directed In Vivo Angiogenesis Assay (DIVAA™) is an in vivo system for the study of angiogenesis that provides quantitative and reproducible results. With the onset of angiogenesis, cellular vascularization proceeds to invade the angioreactor, and as early as nine days post-implantation, there are enough cells to determine an effective dose response to angiogenic modulating factors.

This definition also includes that each of the components of the composition is present in subtherapeutic amounts, i.e., that the amount of each component alone is not sufficient for the desired therapeutic success. However, both components together may result in the desired therapeutic success.

Alternatively, it is also envisaged that each of the components is itself present in an amount sufficient for the desired therapeutic success.

“Therapeutically effective combinations” are thus generally combined amounts of antiangiogenic agents and viruses or viral agents that function to potentiate themselves in their level of cell killing.

“Malignant cells” or “malignant neoplasic cells” stem from tumors or are capable of forming tumors that describe a clinical course that progresses rapidly to death. The term is typically applied to neoplasms that show aggressive behavior characterized by local invasion or distant metastasis.

“Benign neoplastic cells” can refer to any medical condition which, untreated or with symptomatic therapy, will not become life-threatening. It is used in particular in relation to tumors, which may be benign or malignant. Benign tumors do not invade surrounding tissues and do not metastasize to other parts of the body. The word is slightly imprecise, as some benign tumors can, due to mass effect, cause life-threatening complications. The term therefore applies mainly to their biological behavior. Still tumors may be benign but at risk for degeneration into malignancy. These are termed “premalignant”.

The terms “contacted” and “exposed”, when applied to a cell, are used interchangeably to describe the process by which a virus, such as an adenovirus or a herpesvirus, and an antiangiogenic compound are delivered to a target cell or are placed in direct juxtaposition with the target cell. To achieve cell killing, both agents are delivered to a cell in a combined amount effective to kill the cell, i.e., to induce programmed cell death or apoptosis.

The terms “killing”, “programmed cell death” and “apoptosis” are used interchangeably in the present text to describe a series of intracellular events that lead to target cell death.

As used herein a “pharmaceutical composition” means compositions that may be formulated for in vivo administration by dispersion in a pharmacologically acceptable solution or buffer.

As used herein the term “replication-competent” virus refers to a virus that produces infectious progeny in infected cells, at least in certain cells such as cancer cells.

As used herein the term “plaque-forming unit” (pfu) means one infectious virus particle.

As used herein, the term “oncolytic” and “oncolytic viruses” refer to cancer killing, i.e. “onco” meaning cancer and “lytic” meaning “killing”. As used herein, where oncolytic refers to an “oncolytic virus” and an “OV,” this virus represents a virus that may kill a cancer cell.

In context of the present invention, the term “antibody molecule” relates to full immunoglobulin molecules, preferably IgMs, IgDs, IgEs, IgAs or IgGs, more preferably IgG1, IgG2a, IgG2b, IgG3 or IgG4 as well as to parts of such immunoglobulin molecules, like Fab-fragments or VL-, VH- or CDR-regions. Furthermore, the term relates to modified and/or altered antibody molecules, like chimeric and humanized antibodies. The term also relates to modified or altered monoclonal or polyclonal antibodies as well as to recombinantly or synthetically generated/synthesized antibodies. The term also relates to intact antibodies as well as to antibody fragments/parts thereof, like, separated light and heavy chains, Fab, Fab/c, Fv, Fab′, F(ab′)2. The term “antibody molecule” also comprises antibody derivatives, the bifunctional antibodies and antibody constructs, like single chain Fvs (scFv), bispecific scFvs or antibody-fusion proteins. Further details on the term “antibody molecule” of the invention are provided herein below.

The term “endothelial cells” means those cells making up the endothelium, the monolayer of simple squamous cells which lines the inner surface of the circulatory system. These cells retain a capacity for cell division, although they proliferate very slowly under normal conditions, undergoing cell division perhaps only once a year. In contrast, in normal vessels the proportion of proliferating endothelial cells is especially high at branch points in arteries, where turbulence and wear seem to stimulate turnover (Goss, 1978). Normal endothelial cells are quiescent i.e., are not dividing and as such are distinguishable from angiogenic endothelial cells as discussed below. Endothelial cells also have the capacity to migrate, a process important in angiogenesis.

Endothelial cells form new capillaries in vivo when there is a need for them, such as during wound repair or when there is a perceived need for them as in tumor formation. The formation of new vessels is termed angiogenesis, and involves molecules (angiogenic factors) which can be mitogenic or chemoattractant for endothelial cells (Klagsbrun and D'Amore, 1991). During angiogenesis, endothelial cells can migrate out from an existing capillary to begin the formation of a new vessel i.e., the cells of one vessel migrate in a manner which allows for extension of that vessel (Speidel, 1933). In vitro studies have documented both the proliferation and migration of endothelial cells; endothelial cells placed in culture can proliferate and spontaneously develop capillary tubes (Folkman and Haudenschild, 1980).

The terms “angiogenic endothelial cells” and “endothelial cells undergoing angiogenesis” and the like are used interchangeably herein to mean endothelial cells (as defined above) undergoing angiogenesis (as defined above). Thus, angiogenic endothelial cells are endothelial cells which are proliferating at a rate far beyond the normal condition of undergoing cell division roughly once a year and can vary greatly depending on factors such as the age and condition of the patient, the type of tumor involved, the type of wound, etc. Provided the difference in the degree of proliferation between normal endothelial cells and angiogenic endothelial cells is measurable and considered biologically significant then the two types of cells are differentiable per the present invention, i.e., angiogenic endothelial cells differentiable from corresponding, normal, quiescent endothelial cells in terms of preferential binding of cationic liposomes.

The term “lipid” is used in its conventional sense as a generic term of organic molecules having a good solubility in organic solvents and no or only a low solubility in water. The term encompasses fats, fatty oils, essential oils, waxes, steroid, sterols, phospholipids, glycolipids, sulpholipids, aminolipids, chromolipids, fatty acids and the alcohol-ether-soluble constituents of protoplasm, which are insoluble in water.

The term “cationic lipid” is used herein to encompass any lipid which will be determined as being cationic due to its positive charge (at physiological pH).

The term “liposome” encompasses any compartment enclosed by a lipid bilayer. Liposomes are also referred to as lipid vesicles. In order to form a liposome the lipid molecules comprise elongated nonpolar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two monolayer, sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane. As used in connection with the present invention, the term liposome includes multilamellar liposomes, which may have a diameter in the range of 1 to 10 micrometers and are comprised of anywhere from two to hundreds of concentric lipid bilayers alternating with layers of an aqueous phase, and preferably includes unilamellar vesicles which are comprised of a single lipid layer and generally have a diameter in the range of about 20 to about 400 nanometers (nm).

Cationic liposomes are liposomes having a positive charge which can be functionally defined as having a zeta potential of greater than 0 mV when present at physiological pH. The determination of the charge refers to the liposomes as prepared for the intended use, and as determined in vitro. A binding of substances that may alter the charge in the in vivo environment is considered by this definition. Cationic liposomes may comprise cationic lipids but are not necessarily entirely composed of cationic lipids.

In a preferred embodiment, the cationic liposome comprises a zeta potential of greater than about +20 mV when measured in about 0.05 mM KCl solution in about 40 mV.

In the context of the present invention the expression “at least” means the combination of one or more different types of oncolytic viruses with one or more antiangiogenic agents. Throughout the invention, preferably one oncolytic virus and one antiangiogenic agent are combined.

Oncolytic viruses are well known in the art. In principle any virus capable of selective replication in neoplastic cells including cells of tumors, neoplasms, carcinomas, sarcomas, and the like may be utilized in the invention. Selective replication in neoplastic cells means that the virus replicates at least 1×10⁴ preferably times 1×10⁵, especially 1×10⁶ more efficient in at least three cell lines established from different tumors compared to cells from at least three different non-tumorigenic tissues.

Oncolytic viruses may additionally or alternatively be targeted to specific tissues or tumor tissues. This can be achieved for example through transcriptional targeting of viral genes (e.g. WO 96/39841) or through modification of viral proteins that are involved in the cellular binding and uptake mechanisms during the infection process (e.g. WO 2004033639 or WO 2003068809).

A wide range of viruses are contemplated as oncolytic viruses in the present invention, such as but not limited to herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SrN) and sendai virus (SV). Tables 1-7 below provide an overview of examples previously published oncolytic viruses (taken from www.oncolyticVirus.org).

TABLE 1
Oncolytic viruses targeting oncogenic ras or defective Interferon pathways.

| Virus (Company, | | | | |
|---|---|---|---|---|
| if known) | Viral gene defect | Cellular Target | Tumor models | References |
| Influenza A | NS1 | PKR | Melanoma | (1) |
| HSV1mutants: | ICP34.5 | Protein | Brain, Colorectal, | (2, 3) |
| R3616, 1716, | | phosphatase 1a, | ovarian, lung, | |
| G207 (Medigene, | | Defective | prostate, breast | |
| Inc.), MGH1 | | interferon | | |
| | | signaling. | | |
| Reovirus | None | Overactive Ras | Brain, ovarian, | (4-7). |
| (Oncolytics | | pathway | breast, colorectal | |
| Biotech., Inc.) | | | | |
| VSV | None | Defective | Melanoma | (8) |
| | | Interferon | | |
| | | signaling | | |
| Newcastle | None | Overactive Ras | Fibrosarcoma, | (9) |
| disease virus | | pathway | Neuroblastoma | |
| (Provirus) | | | | |

TABLE 2
Oncolytic viruses targeting defective p16 tumor suppressor pathways.

| Virus (Company, | | | | | |
|---|---|---|---|---|---|
| if known) | Mutated viral gene | | Cellular target | Effect | References |
| Adenovirus D24 | E1A-CR2 | | PRB | Viral replication | (10, 11) |
| and dl922-947 | domain | | | restricted to pRB- | |
| (Onyx | | | | defective mutants | |
| Pharmaceuticals) | | | | | |
| Adenovirus | E1A-CR1 and | | PRB, p300, p107, | In keratinocytes, | (12) |
| CB106 | CR2 domains | | p130 | viral replication | |
| | | | | restricted to | |
| | | | | papillomavirus | |
| | | | | E6/E7 expressors | |
| Adenovirus | a) | E1A-CR1 | PRB and | Increased | (13) |
| ONYX- | b) | E2F promoter | upregulated E2F | dependence of | |
| 411(Onyx | | driving E1A | transcription | virus replication | |
| Pharmaceuticals) | | and E4 genes | factor | on overactive | |
| | c) | E3 deletion | | E2F | |
| HSV: hrR3, | Ul39 (ICP6) | | RR activity | Viral replication | (14, 15) |
| rRp450, | | | elevating dNTP | depends on dNTP | |
| HSV1yCD, | | | pools | pools | |
| MGH1, G207 | | | | | |
| Medigene, Inc.), | | | | | |
| G47Δ | | | | | |
| HSV Myb34.5 | a) | UL39 (ICP6) | RR activity | Increased viral | (16, 17) |
| (Prestwick | b) | B-Myb (E2F- | elevating dNTP | replicative | |
| Scientific, Inc.) | | responsive) | pools and | dependence on | |
| | | promoter | upregulated E2F | E2F activity | |
| | | driving γ34.5 | transcription | | |
| | | gene) | factor | | |

| | | | | |
|---|---|---|---|---|
| Vaccinia vvDD- | TK gene | Elevated dTTP | Viral replication | (18, 19) |
| GFP | | (due to cellular | restricted to cells | |
| | | TK?) | with dTTP pools | |

TABLE 3
Oncolytic viruses targeting defective p53 tumor suppressor pathway.

| Virus (Company, | | | | | |
|---|---|---|---|---|---|
| if known) | Mutated viral gene | | Cellular target | Effect | References |
| Adenovirus | E1B-55 Kd | | p53 | Viral replication | (20) |
| ONYX-015 | | | | restricted to p53- | |
| (Onyx | | | | defective mutants | |
| Pharmaceuticals) | | | | | |
| Adenovirus | 1) | p53 promoter | p53, p300. | Expression of E2 | (22) |
| 01/PEME (Canji) | | driving | | and subsequent | |
| | | expression of | | viral genes | |
| | | E2F | | dependent on loss | |
| | | antagonist | | of p53 function; | |
| | 2) | E1A-CR1 | | wild-type p53 | |
| | | p300 binding- | | function | |
| | | domain | | enhanced by | |
| | 3) | E3 deletion | | p300 | |
| | 4) | Extra Major | | coactivation; | |
| | | Late | | increased | |
| | | Promoter | | adenoviral | |
| | | driving | | release and cell | |
| | | expression of | | death by | |
| | | E3-11.6 Kd | | adenoviral death | |
| | | | | protein (21) | |

| | | | | |
|---|---|---|---|---|
| AAV | AAV unusual | p53/p21 | Lack of G2/M | (23) |
| | DNA structure is | | arrest in p53- | |
| | precipitating | | defective cells, | |
| | factor | | infected with | |
| | | | AAV, causes cell | |
| | | | death | |

TABLE 4
Targeting of oncolytic viruses with tumor-specific promoters.

| Virus (Company, | Tumor-specific | | | | |
|---|---|---|---|---|---|
| if known) | Promoter | | Viral gene | Effect | References |
| Adenovirus | PSA (prostate) | | E1A | Replication | (24) |
| CV706 (Calydon, | | | | restricted to | |
| Inc.) | | | | prostate tissue | |
| Adenovirus | a) | Rat probasin | E1A and E1B | Same as above | (25, 26) |
| CN787 (Calydon, | | promoter for | | | |
| Inc.) | | E1A | | | |
| | b) | PSA for E1B | | | |

| | | | | |
|---|---|---|---|---|
| Adenovirus | AFP | E1A and E1B | Replication | (27) |
| CV980 (Calydon, | (hepatocellular | | restricted to | |
| Inc.) | carcinoma) | | hepatic tumors. | |
| Adenovirus | E2F1 promoter | E1A and E4 | Increased | (13) |
| ONYX-411 | (most tumors) | | dependence of | |
| (Onyx | | | virus replication | |
| Pharmaceuticals) | | | on overactive | |
| | | | E2F | |
| Adenovirus | p53 promoter | E2F antagonist. | Expression of E2 | (22) |
| 01/PEME (Canji | (most tumors) | | and subsequent | |
| Inc.) | | | viral genes | |
| | | | dependent on loss | |
| | | | of p53 function | |
| CG8840 (Cell | Uroplakin II | E1A and E1B | Replication | (28) |
| Genesys, Inc.) | (bladder) | | restricted to | |
| | | | bladder cancer | |
| KD1-SPB | Surfactant protein | E4 | Replication | (29) |
| | B | | improved in lung | |
| | | | tumors | |
| HSV Myb34.5 | B-Myb promoter | g34.5 (ICP34.5) | Improved | (16, 17) |
| (Prestwick | (most tumors) | | replication in | |
| Scientific, Inc.) | | | tumors | |
| HSV DF3g34.5 | DF3 promoter | g34.5 (ICP34.5) | Improved | (30) |
| | | | replication in | |
| | | | MUC1-positive | |
| | | | pancreatic and | |
| | | | breast tumor | |
| | | | cells. | |
| HSV G92A | Albumin | ICP4 | Replication | (31) |
| | promoter | | restricted in | |
| | | | hepatoma | |

TABLE 5
Targeting with “tumor-selective” infection.

| | Redirected viral | | | |
|---|---|---|---|---|
| Virus | ligand | Cellular target | Effect | References |
| Dual Adenovirus | Bispecific- | EGFR | Redirects viral | (32) |
| system: | antibody binding | | infection to | |
| AdsCAR-EGF + | adenovirus fiber | | EGFR-expressing | |
| Δ24 | to EGFR | | cells | |
| Adenovirus: | H1-loop in Fiber | Integrin | Redirects viral | (33) |
| Ad5-D24RGD | of Ad modified | | infection to | |
| | by incorporation | | integrin- | |
| | of RGD | | expressing cells. | |
| D24 or ONYX- | Infusion of | EGFR | Redirects viral | (34) |
| 015 | bispecific | | infection to | |
| | antibodies to | | EGFR-expressing | |
| | fiber and EGFR | | cells | |
| Ad 5/35 | Fiber of | Unknown | Redirects viral | (35) |
| | adenovirus | | infection away | |
| | serotype 35 | | from CAR and | |
| | substituted into | | towards an | |
| | adenovirus | | unidentified | |
| | serotype 5 | | cellular receptor | |
| | | | present in human | |
| | | | breast cancer | |

TABLE 6
Other mechanisms of oncolytic virus targeting.

| | Defect in viral | | Oncolytic | |
|---|---|---|---|---|
| Virus | gene | Effect | mechanism | References |
| Vaccinia vvDD- | Vaccinia Growth | Cannot prime | Only dividing | (18) |
| GFP | Factor | neighboring cells | tumor cells will | |
| | | to divide | replicate, because | |
| | | | normal cells are | |
| | | | not “primed” by | |
| | | | VGF | |
| Poliovirus | Substitutes | Loss of | Tumor cells can | (36) |
| PV1(RIPO) | poliovirus IRES | neurovirulence, | still propagate | |
| | element with | because neurons | virus | |
| | rhinovirus 2 | cannot translate | | |
| | IRES | mRNA with | | |
| | | substituted IRES | | |
| Adenovirus E1- | E1 | Does not | Tumor cells can | (37, 38) |
| | | replicate | complement the | |
| | | | E1 defect | |
| Adenovirus | E1 defect with | DNA replication | Adenoviral | (39) |
| Ad.IR-BG | inverted repeats | rearranges the | replication only | |
| | flanking reporter | construct so that | occurs in tumor | |
| | transgene in | promoter is 5′ to | cells that | |
| | antisense | reporter | complement the | |
| | orientation to | transgene | E1 defect | |
| | promoter | | | |
| Measles, mumps, | None | Tumor lysis | Unknown | (40, 41) |
| Sindbis, Sendai | | | | |

TABLE 7
Oncolytic viruses that express anti-cancer cDNAs.

| | Viral gene | Anticancer | Prodrug > | | |
|---|---|---|---|---|---|
| Virus | defect | cDNA | Metabolite | Effect | Reference |
| HSV1: hrR3, | ICP6 | TK | Ganciclovir > GCV- | Predominant | (42-49) |
| MGH1, G207 | and/or | | Phosphate | anticancer action | |
| (Medigene, Inc.) | ICP34.5 | | | in some situations, | |
| | | | | but increased | |
| | | | | antiviral action in | |
| | | | | others (FIG. 5) | |
| HSV1: rRp450 | ICP6 | CYP2B1 | Cyclophosphamide > | Predominant | (15) |
| | | | Phosphoramide | anticancer action + | |
| | | | Mustard | immunosuppressive | |
| | | | | effects. | |
| Adenovirus: | E1B55kD | Fused TK- | Ganciclovir > | Combination of | (50) |
| FGR | | CD gene | GCV-Phosphate + | FGR, GCV, 5FC | |
| | | | 5-fluorocytosine > | and radiation | |
| | | | 5fluorouracil | shows | |
| | | | | predominant | |
| | | | | anticancer action | |
| HSV1: Fu-10 | Unknown | Fusogenic | Not applicable | Enhanced fusion | (51) |
| | | glycol- | | of cell membranes | |
| | | protein | | caused by | |
| | | | | replicating virus | |
| | | | | increases | |
| | | | | anticancer effect | |
| Adenovirus: | E3 | Interferon | Not applicable | Increased | (52) |
| ad5/IFN | | | | anticancer effect | |
| | | | | compared to | |
| | | | | control E3-deleted | |
| | | | | adenovirus | |
| Adenovirus; | E1B55KD | TK | Ganciclovir > | Contradictory | (53, 54) |
| Ad.TK^{RC}, Ad.OW34 | | | GCV-Phosphate | anticancer effects | |
| Adenovirus: | E3-19K | TK | Ganciclovir > | Increased | (55) |
| Ig.Ad5E1⁺.E3TK | | | GCV-Phosphate | anticancer effect | |
| | | | | in glioma | |
| HSV1: Mix of | ICP6 and | IL2 | Not applicable | At low dose, the | (56) |
| G207 + | ICP34.5 | | | mix was more | |
| Defective HSv- | | | | effective than | |
| IL2 | | | | either virus alone | |
| HSV1: NV1042 | Complex | IL12 | Not applicable | Increased | (57) |
| | | | | anticancer effect | |
| HSV1: Mix of | ICP6 and | Soluble | Not applicable | Increased | (58) |
| G207 + | ICP34.5 | B7-1 | | anticancer effect | |
| Defective HSV- | | | | | |
| soluble B7-1 | | | | | |
| HSV1: | ICP6 | Yeast | 5-fluorocytosine > | Increased | (59) |
| HSV1yCD | | cytosine | 5-fluorouracil | anticancer effect | |
| | | deaminase | | minimal antiviral | |
| | | | | effect | |
| Vaccinia: VCD | TK | Bacterial | 5-fluorocytosine > | Increased effect at | (60) |
| | | cytosine | 5-fluorouracil | low viral dose | |
| | | deaminase | | | |
| HSV1 | ICP34.5 | IL4, IL12, | Not applicable | Increased | (61, 62) |
| | | IL10 | | anticancer effect | |
| | | | | for IL12 and IL4, | |
| | | | | but antagonistic | |
| | | | | effect for IL10 | |

Tables 1-7 taken from http://www.oncolyticvirus.org/.

### LIST OF REFERENCES OF TABLES 1-7

1. Bergmann M et al., 2001, Cancer Res, 61: 8188-8193;
2. Leib, D A et al., 2000, Proc Natl Acad Sci 97: 6097-6101;
3. Farassati, F et al., 2001, Nat Cell Biol, 3: 745-750;
4. Strong, J E et al. 1998, Embo J, 17: 3351-3362;
5. Coffey, M C et al., 1998, Science, 282: 1332-1334;
6. Norman, K L et al, 2002, Hum Gene Ther, 13: 641-652;
7. Wilcox, M E et al., 2001, J Natl Cancer Inst, 93: 903-912;
8. Stojdl, D F et al., 2000 Nat Med, 6: 821-825;
9. Lorence, R M et al., 1994, J Natl Cancer Inst, 86: 1228-1233;
10. Fueyo, J et al., 2000, Oncogene, 19: 2-12;
11. Heise, C. et al., 2000, Nat Med, 6: 1134-1139;
12. Balague, C. et al., 2001, J Virol, 75: 7602-7611;
13. Johnson, L. et al., 2002, Cancer Cell, 1: 325-337;
14. Carroll, N M. et al., 1996, Ann Surg, 224: 323-329; discussion 329-330;
15. Chase, M. et al., 1998, Nat Biotechnol, 16: 444-448;
16. Chung, R Y et al., 1999, J Virol, 73: 7556-7564;
17. Nakamura, H et al., 2002, J Clin Invest, 109: 871-882;
18. McCart, J A et al., 2001, Cancer Res, 61: 8751-8757;
19. Puhlmann, M et al., 1999, Hum Gene Ther, 10: 649-657;
20. Bischoff, J R et al., 1996, Science, 274: 373-376;
21. Tollefson, A E et al., 1996, J Virol, 70: 2296-2306;
22. Ramachandra, M et al., 2001, Nat Biotechnol, 19: 1035-1041;
23. Raj, K et al., 2001, Nature, 412: 914-917;
24. Rodriguez, R et al., 1997, Cancer Res, 57: 2559-2563;
25. Yu, D C et al., 1999, Cancer Res, 59: 4200-4203;
26. Chen, Y et al., 2001, Cancer Res, 61: 5453-5460;
27. Li, Y et al., 2001, Cancer Res, 61: 6428-6436;
28. Zhang, J et al., 2002, Cancer Res, 62: 3743-3750;
29. Doronin, K et al., 2001, J Virol, 75: 3314-3324;
30. Mullen, J T et al. Annals of Surgery, in press;
31. Miyatake, S I et al., 1999, Gene Ther, 6: 564-572;
32. Hemminki, A et al., 2001, Cancer Res, 61: 6377-6381;
33. Dmitriev, I et al., 1998, J Virol, 72: 9706-9713;
34. van der Poel, H G et al., 2002, J Urol, 168: 266-272;
35. Shayakhmetov, D M et al., 2002, Cancer Res, 62: 1063-1068;
36. Gromeier, M. et al., 2000, Proc Natl Acad Sci, 97: 6803-6808;
37. Nevins, J R, 1981, Cell, 26: 213-220;
38. Steinwaerder, D S et al., 2000, Hum Gene Ther, 11: 1933-1948;
39. Steinwaerder, D S et al., 2001, Nat Med, 7: 240-243;
40. Grote, D et al., 2001, Blood, 97: 3746-3754;
41. Asada, T, 1974, Cancer, 34: 1907-1928;
42. Boviatsis, E J et al., 1994, Cancer Res, 54: 5745-5751;
43. Kramm, C M et al., 1996, Hum Gene Ther, 7: 1989-1994;
44. Kasuya, H et al., 1999, J Surg Oncol, 72: 136-141;
45. Kramm, C M et al., 1997, Hum Gene Ther, 8: 2057-2068;
46. Carroll, N M et al., 1997, J Surg Res, 69: 413-417;
47. Yoon, S S et al., 1998, Ann Surg, 228: 366-374;
48. Todo, T et al., 2000, Cancer Gene Ther, 7: 939-946;
49. Samoto, K et al., 2002, Neurosurgery, 50: 599-605; discussion 605-596;
50. Freytag, S O et al., 1998, Hum Gene Ther, 9: 1323-1333;
51. Fu, X. and Zhang, X., 2002, Cancer Res, 62: 2306-2312;
52. Zhang, J F et al., 1996, Proc Natl Acad Sci 93: 4513-4518;
53. Wildner, O et al., 1999, Cancer Res, 59: 410-413;
54. Morris, J C and Wildner, O, 2000, Mol Ther, 1: 56-62;
55. Nanda, D et al., 2001, Cancer Res, 61: 8743-8750;
56. Zager, J S et al., 2001, Mol Med, 7: 561-568;
57. Wong, R J et al., 2001, Hum Gene Ther, 12: 253-265;
58. Todo, T et al., 2001, Cancer Res, 61: 153-161;
59. Nakamura, H et al., 2001. Cancer Res, 61: 5447-5452;
60. McCart, J A, 2000, Gene Ther, 7: 1217-1223;
61. Andreansky, S et al., 1998, Gene Ther, 5: 121-130;
62. Parker, J N et al., 2000, Proc Natl Acad Sci 97: 2208-2213;
63. Pechan, P A et al., 1996, Hum Gene Ther, 7: 2003-2013; and
64. Meignier, B. et al., 1988, J Infect Dis, 158: 602-614, all incorporated by reference.

In a preferred embodiment, said oncolytic virus is selected from the group consisting of herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SIN) and sendai virus (SV).

In one embodiment viruses are used that show per se selective replication in neoplastic cells. One examples for such virus is reovirus.

Preferably, said oncolytic virus is an herpes virus, more preferably selected from the group consisting of (i) herpes simplex virus type 1 (HSV-1), i.e. a herpes virus that causes cold sores and fever, (ii) herpes simplex virus type 2 (HSV-2), which is the genital herpes, (iii) herpes zoster or varicella zoster virus, i.e. a herpes virus that causes chickenpox and shingles, (iv) Epstein-Barr virus (EBV), i.e. a herpes virus that causes infectious mononucleosis; associated with specific cancers like Burkitt's lymphoma and nasopharyngeal carcinoma, (ν) cytomegalovirus (CMV), any of a group of herpes viruses that enlarge epithelial cells and can cause birth defects and can affect humans with impaired immunological systems.

More preferably, said oncolytic virus is a herpes simplex virus, even more preferably herpes simplex virus 1 (HSV-1) or herpes simplex virus 2 (HSV-2).

In a preferred embodiment, said herpes virus is an attenuated virus, especially an attenuated herpes virus.

In the context of the present invention, the term “attenuated” means that the respective virus is modified to be less virulent or ideally non-virulent in normal tissues. In a preferred embodiment this modification/attenuation does not or only minimally effect its ability to replicates in tumor, especially in neoplastic-cells and therefore increases its usefulness in therapy.

In a further preferred embodiment, said attenuated HSV-1 has a deletion of an inverted repeat region of the HSV genome such that the region is rendered incapable of expressing an active gene product from one copy only of each of α0, α4, ORFO, ORFP, and γ₁34.5. Preferably, said attenuated HSV-1 is NV1020. Further examples are NV1023 and NV1066.

NV1020 is a non-selected clonal derivative from R7020, a candidate HSV-1/2 vaccine strain that was obtained from Dr. B. Roizman (Meignier et al., 1998). The structure of NV1020 is characterized by a 15 kilobase deletion encompassing the internal repeat region, leaving only one copy of the following genes, which are normally diploid in the HSV-1 genome: ICPO, ICP4, the latency associated transcripts (LATs), and the neurovirulence gene, γ₁34.5. A fragment of HSV-2 DNA encoding several glycoprotein genes was inserted into this deleted region. In addition, a 700 base pair deletion encompasses the endogenous thymidine kinase (TK) locus, which also prevents the expression of the overlapping transcripts of the U_{L}24 gene. An exogenous copy of the HSV-1 TK gene was inserted under control of the Δ4 promotor.

Especially preferred are Herpes simplex virus type 1 (HSV-1) mutants attenuated for neurovirulence which are in clinical development for the treatment of various cancer diseases. Such mutants are described in the publications cited above and are derived from known laboratory strains such as strain F, strain 17 or strain KOS, but also from clinical isolates.

According to a further preferred embodiment of the invention, said attenuated virus, preferably herpes simplex virus, especially HSV-1 is rendered incapable of expressing an active gene product by nucleotide insertion, deletion, substitution, inversion and/or duplication.

The virus may be altered by random mutagenesis and selection for a specific phenotype as well as genetic engineering techniques. Methods for the construction of engineered viruses are known in the art and e.g. described in Sambrook et al., 1989, and the references cited therein. Virological considerations are also reviewed in Coen, 1990, and the references cited therein. References drawn specifically to HSV-1 include: Geller and Breakefield, 1988; Geller and Freese, 1990, Geller, 1988, Breakefield and Geller, 1987; Shih et al., 1985; Palella et al., 1988, Matz et al., 1983; Smiley 1980, Mocarski et al., 1980; Coen et al., 1986.

Examples for mutations rendering herpes simplex virus incapable of expressing at least one active gene product include point mutations (e.g. generation of a STOP codon), nucleotide insertions, deletions, substitutions, inversions and/or duplications.

According to a preferred embodiment of the invention, said attenuated herpes simplex virus, preferably HSV-1, is rendered incapable of expressing an active gene product from both copies of γ₁34.5. Specific examples for said mutants are R3616, 1716, G207, MGH-1, SUP, G47Δ, R47Δ, JS1/ICP34.5-/ICP47- and DM33.

Preferably, said herpes simplex virus is further mutated in one or more genes selected from U_{L}2, U_{L}3, U_{L}4, U_{L}10, U_{L}11, U_{L}12, U_{L}12.5, U_{L}13, U_{L}16, U_{L}20, U_{L}21, U_{L}23, U_{L}24, U_{L}39 (large subunit of ribonucleotide reductase), U_{L}40, U_{L}41, U_{L}43, U_{L}43.5, U_{L}44, U_{L}45, U_{L}46, U_{L}47, U_{L}50, U_{L}51, U_{L}53, U_{L}55, U_{L}56, α22, U_{S}1.5, U_{S}2, U_{S}3, U_{S}4, U_{S}5, U_{S}7, U_{S}8, U_{S}8.5, U_{S}9, U_{S}10, U_{S}11, Δ47, Ori_{S}TU, and LATU, preferably U_{L}39, U_{L}56 and α47,

According to an especially preferred embodiment, said attenuated HSV-1 is G207 or G47Δ.

Especially preferred are further mutations in U_{L}39 (large subunit of ribonucleotide reductase), U_{L}56 and/or α47. Examples for such attenuated HSV-1 are G207, G47Δ, R47Δ, JS1/ICP34.5-/ICP47-, MGH-1, SUP and DM33.

G207 (as described in U.S. Pat. No. 5,585,096) is incapable of expressing both (i) a functional γ₁34.5 gene product and an active ribonucleotide reductase (ICP6). G207 replicates in neoplastic cells, effecting a lytic infection with consequent cell death, but is highly attenuated in non-dividing cells, thereby targeting viral spread to tumors. G207 is non-neuropathogenic, causing no detectable disease in mice and non-human primates (Mineta et al., 1995).

The conditionally replicating HSV-1 vector G47 has been constructed by deleting the α47 gene and the promoter region of US11 from G207 (WO 02076216, Todo et al., 2001).

Further attenuated mutants can easily produced e.g. by applying the procedures to generate recombinant viruses as described by Post and Roizman (1981), and U.S. Pat. No. 4,769,331.

Methods for producing and purifying the oncolytic virus used according to the invention are described in the publications cited above. Generally, the virus may be purified to render it essentially free of undesirable contaminants, such as defective interfering viral particles or endotoxins and other pyrogens, so that it will not cause any undesired reactions in the cell, animal, or individual receiving the virus. A preferred means of purifying the virus involves the use of buoyant density gradients, such as cesium chloride gradient centrifugation.

In a preferred embodiment, the oncolytic virus, preferably the herpes simplex virus further contains foreign DNA, i,e DNA which is not derived from said virus.

This foreign DNA may be a heterologous promoter region, a structural gene, or a promoter operatively linked to such a gene. Representative promoters include, but are not limited to, the CMV promoter, LacZ promoter, Egr promoter or known HSV promoters. In a preferred embodiment, the structural gene is selected from the group of a cytokine/chemokine, a suicide gene, a fusogenic protein or a marker gene. Preferred cytokines/chemokines are IL-4, IL-12 and GM-CSF. Preferred suicide genes are p450 and cytosine deaminase. A fusogenic protein is for example Gibbon ape leukemia virus envelope. Common marker genes are GFP or one of its variants and LacZ.

In a further preferred embodiment the oncolytic virus is further modified to have an altered host cell specificity. Such mutants are for example known for HSV-1 from WO 2004/033639, US 2005271620, Kamiyama et al. (2006) and Menotti et al. (2006). Here, glycoproteins of HSV-1 such as gD, gC are fused to a ligand, especially to single-chain antibodies, that specifically bind to target cells of choice. Further, to detarget such viruses from their natural receptors and heparin sulfate proteoglycan deletions and/or point mutations are made in gB, gC and/or gD (WO 2004/033639, Zhou and Roizman, 2006).

The second component of the combination of the present invention is an antiangiogenic agent.

According to a preferred embodiment, said antiangiogenic agent is selected from the group consisting of agents that target the vascular endothelial growth factor (VEGF) pathway, an integrin, a matrix metalloproteinase (MMP) and/or protein kinase C beta (PKCβ), or a combination thereof.

Vascular endothelial growth factor (VEGF)-mediated angiogenesis is thought to play a critical role in tumor growth and metastasis. Consequently, anti-VEGF therapies may be anti-cancer treatments, either as alternatives or adjuncts to conventional chemo or radiation therapy. Several approaches to targeting VEGF have been investigated. The most common strategies have been receptor-targeted molecules and VEGF-targeting molecules.

Therefore, preferably, said VEGF pathway targeting agent is:
i) an antibody or a fragment thereof against a member of the VEGF family (VEGF, placental growth factor (P1GF), VEGF-B, VEGF-C, VEGF-D) or their receptors (VEGFR-1 (Flt-1), -2 (Flk-1/Kdr), -3 (Flt-4)), and/or
ii) a small molecule tyrosine kinase inhibitor of VEGF receptors, and/or
iii) a soluble VEGF receptor, and/or
iv) a ribozyme which specifically targets VEGF mRNA (Cardones and Banez, 2006).

Preferably, said antibody is a monoclonal antibody, even more preferred Bevacizumab (Avastin), 2C3, or HuMV833 or a combination thereof.

The humanized monoclonal antibody Bevacizumab (Avastin™, Genentech) is approved as an anti-angiogenic agent for treatment of cancer (Wakelee and Schiller, 2005). Bevacizumab is preferably administered to human patients intravenously, and is usually administered in an intravenous infusion of 5 mg/kg every 14 days. The therapy usually is not initiated for at least 28 days following major surgery. It is recommended that the surgical incision is fully healed prior to initiation of bevacizumab therapy (Avastin IV in PDR 60. edition, 2006, Thomson, page 1229-1232).

Other examples of anti-VEGF antibodies, suitable for use in this invention, include 2C3, or HuMV833. 2C3 blocks the interaction of VEGF with VEGFR2 and inhibited tumor growth in mice (Zhang et al., 2002). It is discussed as a promising anti-angiogenic agent and a tumor vascular targeting agent in man (Brekken and Thorpe, 2001). HuMV833 is a humanized form of MV833, a murine monoclonal anti-VEGF antibody that showed activity against a variety of tumors in pre-clinical models. Its administration inhibited growth of melanoma and rhabdomyosarcoma xenografts (Kim et al., 1993). In a phase I clinical trial the recombinant humanized IgG4 anti-VEGF monoclonal antibody was tested to be safe, lack toxicity and to possess some clinical activity in patients with advanced cancer (Jayson et al., 2005).

Several small molecule tyrosine kinase inhibitors, preferably of the VEGF receptor and EGF receptor family have now reached clinical trials. They are of special interest in combination therapy and may be used according to the present invention, since despite high doses often only limited efficacies could be reached.

Consequently, according to a preferred embodiment, said tyrosine kinase inhibitor is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, gefitinib (Iressa®), erlotinib (Tarceva®), EKB-569, HKI-272, and CI-1033, preferably wherein the tyrosine kinase inhibitor is ZD6474.

Sunitinib malate is an oral multitargeted tyrosine kinase inhibitor with antitumor and antiangiogenic activity that recently received approval from the FDA for the treatment of advanced renal cell carcinoma and of gastrointestinal stromal tumors after disease progression on or intolerance to imatinib mesilate therapy (Motzer et al., 2006). Sunitinib (SU11248; Sutent®) has also demonstrated promising clinical activity in the treatment of other advanced solid tumors.

SU5416 (Z-3-[(2,4-dimethylpyrrol-5-yl)methylidenyl]-2-indolinone, Semaxanib), which was considered the prototype of small molecule tyrosine kinase inhibitors, was the first agents to reach clinical trials as a potent and selective VEGFR-2 inhibitor (O'Donnell et al., 2005). SU6668 is an oral inhibitor of VEGFR, platelet-derived growth factor receptor (PDGFR) and fibroblast growth factor receptor (FGFR). Since even maximum doses of SU6668 given orally in phase I clinical studies only led to low plasma levels efficacy as a single agent was not to be expected (Kuenen et al., 2005).

The oral angiogenesis inhibitor PTK 787/ZK 222584 (PTK/ZK, Vatalanib) blocks all known VEGFR tyrosine kinases, including the lymphangiogenic VEGFR-3, in the lower nanomolar range. From a panel of 100 kinases only PDGFR, c-kit, and c-fms are inhibited in the nanomolar range. PTK/ZK functions as a competitive inhibitor at the ATP-binding site of the receptor kinase (Hess-Stumpp et al., 2005). In randomized phase III trials multitargeted tyrosine kinase inhibitors that block VEGF receptor and other kinases in both endothelial and cancer cells, demonstrated survival benefit in patients with metastatic cancer (Jain et al., 2006).

AEE788, obtained by optimization of the 7H-pyrrolo[2,3-d]pyrimidine lead scaffold, is a potent combined inhibitor of both VEGFR and epidermal growth factor receptor (EGFR) tyrosine kinase family members. In animal models of cancer, oral administration of AEE788 efficiently inhibited growth factor-induced EGFR and ErbB2 phosphorylation, as well as VEGF-induced angiogenesis. Taken together, pre-clinical data indicate that AEE788 has potential as an anticancer agent targeting deregulated tumor cell proliferation as well as angiogenic parameters (Traxler et al., 2004). Consequently, AEE788 is currently in Phase I clinical trials in oncology.

Another agent with early promising results in antitumor activity is ZD6474, an inhibitor of VEGFR and EGFR tyrosine kinase activity (Zakarija and Soff, 2005). For example, ZD6474 improved survival in patients with metastatic non-small cell lung cancer in a randomized clinical trial (Morgensztern and Govindan, 2006). Combination therapy e.g. with radiation improved therapeutic response (Cardones and Banez, 2006).

ZD4190, a substituted 4-anilinoquinazoline, is a potent inhibitor of VEGFR-1 and -2 tyrosine kinase activity. Oral dosing of ZD4190 to mice bearing established human tumor xenografts (breast, lung, prostate, and ovarian) elicited significant antitumor activity (Wedge et al., 2000).

In another preferred embodiment of this invention the small molecule tyrosine kinase inhibitor of VEGFR is AZD2171, GW786034, sorafenib (BAY 43-9006), CP-547,632 or AG013736 (Wakelee and Schiller, 2005).

Another agent with highly potent antitumor activity against established tumors and that can be used in the context of the present invention is YM-359445, an orally bioavailable VEGFR-2 tyrosine kinase inhibitor (Amino et al., 2006).

Further encompassed are other tyrosine kinase inhibitors like gefitinib (Iressa®), erlotinib (Tarceva®), EKB-569, HKI-272, and CI-1033.

Gefitinib (Iressa®) is a small molecule EGF receptor-selective inhibitor of tyrosine kinase activity. It has been the first EGF receptor-targeting drug to be registered in 28 countries worldwide, including the USA, for the third-line treatment of chemoresistant non-small cell lung cancer patients (Ciardiello, 2005). Moreover, the EGF receptor inhibitor erlotinib (Tarceva®) has undergone extensive clinical testing and has established clinical activity in non-small cell lung cancer and other types of solid tumors (Heymach et al., 2006). Together with gefitinib and erlotinib, Cl-1033 is also a tyrosine kinase inhibitor targeting the intracellular domain of the EGF receptor and has been studied in clinical settings alone or in combination with radiation or chemotherapy (Khali et al., 2003).

EKB-569 is a selective irreversible inhibitor of the EGF receptor (Erlichman et al., 2006). Like several inhibitors targeting more than one tyrosine kinase, HKI-272 is a dual-specific kinase inhibitor targeting both, EGF receptor and the related ErbB2 tyrosine kinase (Shimamura et al., 2006).

Preferably, the tyrosine kinase inhibitor is ZD6474.

Preferably, said soluble VEGF receptor is VEGF-Trap, a soluble high-affinity VEGF decoy receptor (Cardones and Banez, 2006).

Preferably, said ribozyme specifically targeting VEGF mRNA is Angiozyme™ (Cardones and Banez, 2006).

According to a further preferred embodiment, said antiangiogenic agents targeting MMPs or integrins are chimeric, humanized or fully human monoclonal antibodies.

According to a preferred embodiment, said antiangiogenic agent targeting a MMP is selected from the group consisting of marimastat, metastat (COL-3), BAY-129566, CGS-27023A, prinomastat (AG-3340), and BMS-275291.

These drugs are all in different stages of clinical development, ranging from phase I to III (Heath and Grochow, 2000. Ramnath and Creaven, 2004).

Preferably, said antiangiogenic agent targeting an integrin is selected from the group consisting of SB-267268, JSM6427, and EMD270179 (the compounds are described in (Wilkinson-Berka et al., 2006), Umeda et al., 2006, and Strieth et al., 2006, respectively). The rational behind this is that alpha(ν)-integrins play an important role in neovascularization.

Furthermore, also other factors, as well as protein kinase C beta (PKCβ) can be targeted.

Preferably, said PKCβ-selective inhibitor is Enzastaurin (LY317615, Graff et al., 2005).

Purified antiangiogenic factors from shark cartilage also showed antiangiogenic and antitumor activity (Cho and Kim, 2002; Drugs, 2004)

Consequently, according to a further preferred embodiment, said antiangiogenic agent is selected from the group consisting of a cationic liposome, a Vascular Targeting Agent (VTA), Neovastat (AE-941), U-995, Squalamine, Thalidomide or one of its immunomodulatory analogs, or a combination thereof.

Preferably, said immunomodulatory analog of Thalidomide is selected from the group consisting of lenalidomide, Revlimid, CC-5013, CC-4047, and ACTIMID. Thalidomide and its immunomodulatory analogs (lenalidomide, Revlimid, CC-5013; CC-4047, ACTIMID) are a novel class of compounds mediating anticancer results observed in humans (Teo, 2005) that can be used in the methods of the present invention.

As discussed above, according to the invention vascular targeting agents (VTAs) may be used. These are e.g. designed to cause a rapid and selective shutdown of the blood vessels of tumors. Unlike other antiangiogenic drugs that inhibit the formation of new vessels, VTAs occlude the pre-existing blood vessels of tumors to cause tumor cell death from ischemia and extensive hemorrhagic necrosis (Thorpe, 2004).

According to a further preferred embodiment, said VTA is a small molecule or a ligand-based agent.

Preferably, said small molecule VTA is selected from the group consisting of combretastatin A-4 disodium phosphate (CA4P), ZD6126, AVE8062, Oxi 4503, DMXAA and TZT1027, preferably the small molecule agent is CA4P.

Preferably, said ligand-based VTA uses an antibody, or an antigen-specific part thereof, peptide or growth factor, that bind selectively to tumor vessels versus normal vessels to indirectly target tumors with agents that occlude blood vessels. The ligand-based VTAs include fusion proteins (e.g., VEGF linked to the plant toxin gelonin), immunotoxins (e.g., monoclonal antibodies to endoglin conjugated to ricin A), antibodies linked to cytokines, liposomally encapsulated drugs, and gene therapy approaches.

It is one embodiment of the present invention, that the antiangiogenic agent and/or vascular targeting agent is a cationic liposomal preparation. This involves injecting such preparation preferably systemically into the circulatory system and more preferably intravenously. Cationic liposomes have the ability to selectively bind to angiogenic vascular endothelial cells. It has been shown that such cationic liposomes alone can inhibit the activation of endothelial cells.

Such cationic liposomal preparation may comprise at least one cationic lipid and at least one neutral and/or anionic lipid. Preferably such preparation comprises cationic lipids in an amount of more than about 30 mol % of total lipid and/or having a zetopotential of at least +20 mV. Preferably, said cationic liposomal preparation comprises 1,2-dioleoyl-3-trimethylammonium propane (DOTAP) and 1,2-dioleoyl-sn-glycero-3-phosphocholine (DOPC).

Cationic liposomes can be used to selectively deliver agents such as cytotoxic or chemotherapeutic agents to angiogenic endothelial cells. Therefore, said cationic liposomal preparation comprises as a preferred embodiment at least one cytoxic or chemotherapeutic agent, preferably at least one antimitotic agent, especially Na-Camptothecin (Saetern et al., 2004 and WO 2004/002454) or a taxane, preferably paclitaxel or a derivative thereof (WO 01/17508 and Kunstfeld et al., 2003).

Liposomes are prepared according to standard technologies (WO 98/40052 and WO 2004/002468).

In a further embodiment, the cationic liposomal preparation may comprise a nucleotide sequence such as DNA which encodes a protein, which when expressed, inhibits angiogenesis. The nucleotide sequence is preferably contained within a vector operably connected to a promoter which promoter is preferably only active in angiogenic endothelial cells or can be activated in those cells by the administration of a compound thereby making it possible to turn the gene on or off by activation of the promoter.

Another object of the invention is to provide cationic liposomes which liposomes are comprised of cationic lipids and compounds which are specifically intended and designed to inhibit angiogenesis which compounds may be water soluble or readily dispersable in water or lipid compatible and incorporated in the lipid layers.

Another object of the invention is to provide a method for selectively affecting angiogenic endothelial cells by delivering a cationic lipid/DNA complex to angiogenic endothelial cells, wherein the DNA is attached to a promoter which is selectively activated within an environment which is preferably uniquely associated with angiogenic endothelial cells, i.e, the promoter is not activated in quiescent endothelial cells.

A feature of the invention is that the cationic liposomes of the invention selectively associate with angiogenic endothelial cells with a much higher preference (five-fold or greater and preferably ten-fold or greater) than they associate with corresponding endothelial cells not involved in angiogenesis.

According to a further preferred embodiment of the invention, the antiangiogenic agent is a receptor antagonist of epidermal growth factor receptor (EGFR) signaling pathway.

As it has been discussed above, cetuximab (Erbitux®), which is an EGFR antagonist, is effective in the treatment of tumors in a combination therapy with HSV. EGFR antagonists and specifically cetuximab function as EGFR tyrosine kinase inhibitor by specifically blocking the epidermal growth factor receptor (EGFR) and, as a consequence, inhibiting tumor growth. Furthermore, it is known in the art that besides a number of other anti-tumor activities, EGFR antagonists and specifically cetuximab are also reported to exert their biological activity via inhibition of angiogenesis (Zhu, 2007).

In the context of the present invention, the term “antagonist” denotes a compound which binds either to the receptor itself or to another protein being in interaction with the receptor and which at least partially inhibits the function of the receptor. Consequently, an antagonist according to the present invention can exert its effects on the receptor either directly or indirectly.

Preferably, said receptor antagonist of epidermal growth factor receptor (EGFR) is an EGFR tyrosine kinase inhibitor, i.e. an inhibitor of the tyrosine kinase activity of the EGFR. In general, tyrosine kinase inhibitors are known in the art and include small molecules and intra- or extracellular antibodies.

In a preferred embodiment, said EGFR tyrosine kinase inhibitor is an anti-EGFR monoclonal antibody, e.g. cetuximab (Erbitux®), panitumumab (Vectibix®), nimotuzumab, matuzumab, zalutuzumab, mAb 806, or IMC-11F8. These antibodies are generally known in the art. Most of them are commercially available.

According to a further preferred embodiment of the invention, the antiangiogenic agent is a tyrosine kinase inhibitor.

As it has been discussed above, cetuximab (Erbitux®), which is an EGFR antagonist, is effective in the treatment of tumors in a combination therapy with HSV. Cetuximab is an EGFR antagonist and a known inhibitor of EGFR tyrosine kinase activity. Furthermore, it is known in the art that agents inhibiting tyrosine kinase activity have anti-angiogenic properties (Sequist, 2007; Zhong and Bowen, 2007).

Preferably, said tyrosine kinase inhibitor is selected from the group consisting of agents that target the vascular endothelial growth factor receptor (VEGFR) pathway, the epidermal growth factor receptor (EGFR) pathway, the platelet-derived growth factor receptor (PDGFR), the fibroblast growth factor receptor (FGFR), ErbB2 or an agent that targets a combination thereof.

In a preferred embodiment, said tyrosine kinase inhibitor is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, gefitinib (Iressa®), erlotinib (Tarceva®), EKB-569, HKI-272, and Cl-1033, preferably wherein the tyrosine kinase inhibitor is ZD6474. These compounds have been explained and defined above.

In a further preferred embodiment, said tyrosine kinase inhibitor is a monoclonal antibody, e.g. Bevacizumab (Avastin), 2C3, HuMV833, cetuximab (Erbitux®), panitumumab (Vectibix®), nimotuzumab (TheraCim®), matuzumab, zalutuzumab, mAb 806, or IMC-11F8. These antibodies are generally known and most of them are commercially available.

In a further aspect, the invention relates to a combination of at least one oncolytic virus and at least one receptor antagonist of epidermal growth factor receptor (EGFR) signaling pathway.

All definitions and further comments given above for the use of at least one receptor antagonist of epidermal growth factor receptor (EGFR) signaling pathway in the context of its antiangiogenic properties also apply to this aspect of the invention.

Preferably, the receptor antagonist is an EGFR tyrosine kinase inhibitor as defined above.

In a preferred embodiment, said EGFR tyrosine kinase inhibitor is an anti-EGFR monoclonal antibody, e.g. cetuximab (Erbitux®), panitumumab (Vectibix®), nimotuzumab, matuzumab, zalutuzumab, mAb 806, or IMC-11F8.

The oncolytic virus as used in the context of this aspect of the invention is the same as defined above.

In a further aspect, the invention relates to a combination of at least one oncolytic virus and at least one tyrosine kinase inhibitor.

All definitions given above for tyrosine kinase inhibitors also apply to this aspect of the invention.

Preferably, said tyrosine kinase inhibitor is selected from the group consisting of agents that target the vascular endothelial growth factor receptor (VEGFR) pathway, the epidermal growth factor receptor (EGFR) pathway, the platelet-derived growth factor receptor (PDGFR), the fibroblast growth factor receptor (FGFR), ErbB2 or an agent that targets a combination thereof.

In a preferred embodiment, said tyrosine kinase inhibitor targets the vascular endothelial growth factor receptor (VEGFR) and is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, Bevacizumab (Avastin®), 2C3, and HuMV833, preferably wherein the tyrosine kinase inhibitor is ZD6474.

In a further preferred embodiment, said tyrosine kinase inhibitor targets epidermal growth factor receptor (EGFR) and is selected from the group consisting of AEE788, ZD6474, gefitinib (Iressa®), erlotinib (Tarceva®), EKB-569, HKI-272, CI-1033, cetuximab (Erbitux®), panitumumab (Vectibix®), nimotuzumab, matuzumab, zalutuzumab, mAb 806, and IMC-11F8.

In a further preferred embodiment, said tyrosine kinase inhibitor targets the platelet-derived growth factor receptor (PDGFR), the fibroblast growth factor receptor (FGFR), ErbB2 or a combination of said receptors, and is selected from the group consisting of SU6668, vatalanib (PTK787/ZK222584) and AEE788.

In a further preferred embodiment, said tyrosine kinase inhibitor is a monoclonal antibody, e.g. Bevacizumab (Avastin®), 2C3, HuMV833, cetuximab (Erbitux®), panitumumab (Vectibix®), nimotuzumab, matuzumab, zalutuzumab, mAb 806, or IMC-11F8.

The oncolytic virus as used in the context of this aspect of the invention is the same as defined above.

An important feature of the invention is that several classes of diseases and/or abnormalities are treated without directly treating the tissue involved in the abnormality e.g., by inhibiting angiogenesis the blood supply to a tumor is cut off and the tumor is killed without directly treating the tumor cells in any manner.

In another aspect, the present invention relates to the use of at least one oncolytic virus for the preparation of a medicament for the treatment of a tumorigenic disease, wherein the oncolytic virus is administered simultaneously, sequentially or separately in combination with an antiangiogenic agent, a receptor antagonist of epidermal growth factor receptor (EGFR) signaling pathway or a tyrosine kinase inhibitor.

Furthermore, the invention also relates to at least one oncolytic virus for use in a method for the treatment of a tumorigenic disease, wherein the oncolytic virus is administered simultaneously, sequentially or separately in combination with at least one antiangiogenic agent, at least one receptor antagonist of epidermal growth factor receptor (EGFR) signaling pathway or at least one tyrosine kinase inhibitor.

In a further aspect, the invention relates to the use of an antiangiogenic agent, a receptor antagonist of epidermal growth factor receptor (EGFR) signaling pathway or a tyrosine kinase inhibitor for the preparation of a medicament for the treatment of a tumorigenic disease, wherein the antiangiogenic agent, the receptor antagonist of epidermal growth factor receptor (EGFR) signaling pathway or the tyrosine kinase inhibitor is administered simultaneously, sequentially or separately in combination with an oncolytic virus.

Furthermore, the invention relates to at least one antiangiogenic agent, at least one receptor antagonist of epidermal growth factor receptor (EGFR) signaling pathway or at least one tyrosine kinase inhibitor for use in a method for the treatment of a tumorigenic disease, wherein the antiangiogenic agent, the receptor antagonist of epidermal growth factor receptor (EGFR) signaling pathway or the tyrosine kinase inhibitor is administered simultaneously, sequentially or separately in combination with at least one oncolytic virus.

Furthermore, the invention relates to the use of the combination of an oncolytic virus and an antiangiogenic agent, a receptor antagonist of epidermal growth factor receptor (EGFR) signaling pathway or a tyrosine kinase inhibitor for the preparation of a medicament for the treatment of a tumorigenic disease, wherein the virus is administered simultaneously, sequentially or separately in combination with the antiangiogenic agent, the receptor antagonist of epidermal growth factor receptor (EGFR) signaling pathway or the tyrosine kinase inhibitor.

Furthermore, the invention relates to a combination of at least one oncolytic virus and at least one antiangiogenic agent, at least one receptor antagonist of epidermal growth factor receptor (EGFR) signaling pathway or at least one tyrosine kinase inhibitor for use in a method for the treatment of a tumorigenic disease, wherein the virus is administered simultaneously, sequentially or separately in combination with the antiangiogenic agent, the receptor antagonist of epidermal growth factor receptor (EGFR) signaling pathway or the tyrosine kinase inhibitor.

All embodiments disclosed above with respect to the oncolytic virus and the antiangiogenic agent also apply to these uses, substances, or combination of the invention.

All embodiments disclosed above with respect to the combination of receptor antagonist of epidermal growth factor receptor (EGFR) signaling pathway or the tyrosine kinase inhibitor on one side and the oncolytic virus on the other side also apply to these uses or substances of the invention.

According to preferred embodiments of theses uses, substances or combination of the invention, the tumor is contacted first with the virus and then with the antiangiogenic agent, the receptor antagonist of epidermal growth factor receptor (EGFR) signaling pathway or the tyrosine kinase inhibitor.

Alternatively, the tumor may also be contacted first with the antiangiogenic agent, the receptor antagonist of epidermal growth factor receptor (EGFR) signaling pathway or the tyrosine kinase inhibitor and then with the virus.

In one embodiment of the invention the time span between the contact with the virus and with the antiangiogenic agent or vice versa is 1 to 28 days, preferably 3 to 14 days, especially 7 days.

In a preferred embodiment the virus is applied more than once, preferably more than twice, especially, at least 4 times.

In a most preferred embodiment patients receive four doses of virus in weekly or biweekly intervals followed by treatment with the antiangiogenic agent after one week of the last virus application.

As discussed above, the invention is directed to the killing a cell or cells, such as a malignant cell or cells, by contacting or exposing a cell or population of cells to one or more antiangiogenic agents and one or more viruses in a combined amount effective to kill the cell(s). The invention has a particular utility in killing malignant cells.

Consequently, compositions, methods and uses are provided for selectively killing neoplastic cells. The method involves infecting neoplastic cells with an altered virus which is capable of replication in neoplastic cells but spares surrounding non-neoplastic tissue. Upon viral infection, the virus destroys infected cells without causing systemic viral infection.

To kill a cell in accordance with the present invention, one would generally contact the cell with at least one antiangiogenic compound and at least one oncolytic virus, such as HSV-1, in a combined amount effective to kill the cell. It is envisioned that the cell that one desires to kill may be first exposed to a virus, and then contacted with the antiangiogenic agent(s), or vice versa. In such embodiments, one would generally ensure that sufficient time elapses, so that the two agents would still be able to exert an advantageously combined effect on the cell. In one embodiment of the invention the time span between the contact with the virus and with the antiangiogenic agent or vice versa is 1 to 28 days, preferably 3 to 14 days, especially 7 days. The dosing and administration techniques and schedules for antiangiogenic agents and anti-cancer viruses are known in the art.

A number of parameters may be used to determine the effect produced by the compositions and methods of the present invention. These parameters include e.g. measuring the size of the tumor either by the use of calipers, or by the use of radiologic imaging techniques, such as computerized axial tomography (CAT) or nuclear magnetic resonance (NMR) imaging. Moreover, the effect on cell killing can also be determined by the observation of net cell numbers before and after exposure to the compositions described herein. In addition to cell survival the response of the cells to this treatment modality may be assessed by a number of in vitro techniques known in the art, such as enzymatic assays of selected biomarker proteins, changes in size of cells or cell colonies grown in culture. Alternatively, one may measure parameters that are indicative of a cell that is undergoing programmed cell death. such as for example, the fragmentation of cellular genomic DNA into nucleoside size fragments.

According to a preferred embodiment, said virus is to be administered to the patient by means of local, local-regional or systemic injection of from about 10⁸ to 10¹¹ plaque-forming units, preferably of from about 10⁸ to 10⁹ plaque-forming units.

Antiangiogenic agents and/or viruses may be administered to the mammal, often in close contact to the tumor, in the form of a pharmaceutically acceptable composition. In accordance with this invention, any conventional route or technique for administering viruses to a subject can be utilized. For examples of routes of administration refer to WO 00/62735. Direct intralesional injection is contemplated, as are other modes such as loco-regional applications, e.g. administration into the hepatic artery, into the bladder, into the prostate or parenteral routes of administration, such as intravenous, percutaneous, endoscopic, intraperitoneal, intrapleural or subcutaneous injection. In certain embodiments, the route of administration may be oral. In a preferred embodiment of this invention, the virus is administered systemically, for example intravenously.

Suitable pharmacologically acceptable solutions include neutralsalme solutions buffered with phosphate, lactate, Tris, NaCl 0.9%, Ringer solution and the like.

The amount of virus to be administered depends, e.g., on the specific goal to be achieved, the strength of any promoter used in the virus, the condition of the mammal (e.g., human) intended for administration (e.g., the weight, age, and general health of the mammal), the mode of administration, and the type of formulation. In general, a therapeutically or prophylactically effective dose of, e.g., from about 10¹ to 10¹¹ pfu for example, from about 10⁸ to 10¹¹ pfu, e.g., from about 10⁸ to about 10⁹ pfu, although the most effective ranges may vary from host to host, as can readily be determined by one of skill in this art. Also, the administration can be achieved in a single dose or repeated at intervals, as determined to be appropriate by those of skill in this art.

Preferably, said tumorigenic disease is selected from the group consisting of astrocytoma, oligodendroglioma, meningioma, neurofibroma, glioblastoma, ependymoma, Schwannoma, neurofibrosarcoma, neuroblastoma, pituitary adenoma, medulloblastoma, head and neck cancer, melanoma, prostate carcinoma, renal cell carcinoma, pancreatic cancer, breast cancer, lung cancer, colon cancer, gastric cancer, bladder cancer, liver cancer, bone cancer, rectal cancer, ovarian cancer, sarcoma, gastric cancer, esophageal cancer, cervical cancer, fibrosarcoma, squamous cell carcinoma, neurectodermal, thyroid tumor, Hodgkin's lymphoma, non-Hodgkin's lymphoma, hepatoma, mesothelioma, epidermoid carcinoma, and tumorigenic diseases of the blood, preferably wherein said tumorigenic disease is glioblastoma.

According to a further preferred embodiment, said treatment involves the treatment of metastasis of said tumorigenic disease, preferably liver metastasis from colorectal cancer.

In accordance with this invention, any neoplasm can be treated, including but not limited to the following: astrocytoma, oligodendroglioma, meningioma, neurofibroma, glioblastoma, ependymoma, Schwannoma, neurofibrosarcoma, neuroblastoma, pituitary adenoma, medulloblastoma, head and neck cancer, melanoma, prostate carcinoma, renal cell carcinoma, pancreatic cancer, breast cancer, lung cancer, colon cancer, gastric cancer, bladder cancer, liver cancer, bone cancer, rectal cancer, ovarian cancer, sarcoma, gastric cancer, esophageal cancer, cervical cancer, fibrosarcoma, squamous cell carcinoma, neurectodermal, thyroid tumor, Hodgkin's lymphoma, non-Hodgkin's lymphoma, hepatoma, mesothelioma, epidermoid carcinoma, and tumorigenic diseases of the blood, preferably wherein said tumorigenic disease is glioblastoma. In addition, this invention comprises the treatment of metastasis of said tumorigenic diseases, preferably liver metastasis from colorectal cancer.

In particular, metastasis is suppressed using the methods, uses, substances, combinations and compositions of the invention.

In a preferred embodiment of the invention, said treatment is combined with chemotherapy and/or radiotherapy.

Preferably, said further active chemotherapeutic agent is selected from the group consisting of
(i) an alkylating agent including busulfan, carmustine, chlorambucil, cyclophosphamide (i.e., cytoxan), dacarbazine, ifosfamide, lomustine, mecholarethamine, melphalan, platinum containing compounds like cisplatin and carboplatin, procarbazine, streptozocin, and thiotepa, preferably platinum containing compounds like cisplatin and carboplatin.
(ii) an antineoplastic agent including antimitotic agents like paclitaxel or a derivative thereof, bleomycin, dactinomycin, daunorubicin, doxorubicin, idarubicin, mitomycin (e.g., mitomycin C), mitoxantrone, pentostatin, and plicamycin, preferably antimitotic agents like paclitaxel or a derivative thereof,
(iii) an RNA/DNA antimetabolite including fluorodeoxyuridine, capecitabine, cladribine, cytarabine, floxuridine, fludarabine, fluorouracil. gemcitabine, hydroxyurea, mercaptopurine, methotrexate, and thioguanine, preferably 5-fluorouracil (5FU) or capecitabine,
(iv) a natural source derivative including docetaxel, etoposide, irinotecan, paclitaxel, teniposide, topotecan, vinblastine, vincristine, vinorelbine, taxol, prednisone, and tamoxifen, and
(v) an additional chemotherapeutic agent including asparaginase, mitotane, leucovorin, oxaliplatin, DNA topoisomerase inhibiting agents like camptothecin, and anthracyclines like doxorubicin.

Preferably, the chemotherapeutic agent is or comprises oxaliplatin and/or irinotecan.

Preferably, the chemotherapeutic agent is FOLFOX (5-fluoruracil, leucovorin and oxaliplatin) or FOLFIRI (5-fluoruracil, leucovorin and irinotecan), that are currently standard first-line regimens for metastatic colorectal cancer. The addition of bevacizumab prolongs median survival from 12 to 20 months (Goldberg, 2005). FOLFOX is consisting of concurrent treatment with 5-FU, leucovorin (LV, folinic acid), and oxaliplatin. Patients typically receive a treatment every two weeks, all drugs are administered intravenously. LV and oxaliplatin are administered as an infusion lasting two hours, this is followed by 5-FU which is administered in two different ways: a bolus injection lasting a few minutes and a continuous infusion lasting 48 hours. In FOLFIRI intravenously administered 5-FU and LV are combined with irinotecan instead of oxaliplatin. This combination of three drugs is characterized by lower toxicity than FOLFOX making it the preferred 1st-line therapy in advanced colorectal cancer.

Methods for administration of chemotherapeutic drugs are well known in the art and vary depending on, for example, the particular drug (or combination of drugs) selected, the cancer type and location, and other factors about the patient to be treated (e.g., the age, size, and general health of the patient). Any of the drugs listed above, or other chemotherapeutic drugs that are known in the art, are administered in conjunction with the mutant Herpes viruses and antiangiogenic agents described herein.

According to a preferred embodiment of the present invention, said radiation therapy uses photon radiation (electromagnetic energy) like X-rays and gamma rays (including the gamma-knife), internal radiotherapy, intraoperative irradiation, particle beam radiation therapy, and radioimmunotherapy.

Radiotherapy, also called radiation therapy, is the treatment of cancer and other diseases with radiation, typically ionizing radiation. Radiotherapy may be used to treat localized solid tumors, as well as leukemia and lymphoma.

One type of radiation therapy commonly used involves photons (electromagnetic energy). X-rays were the first form of photon radiation to be used to treat cancer. Depending on the amount of energy they possess, the rays can be used to destroy cancer cells on the surface of or deeper in the body. Linear accelerators and betatrons are machines that produce x-rays of increasingly greater energy. The use of machines to focus radiation (such as x-rays) on a cancer site is called external beam radiotherapy.

Gamma rays are another form of photons used in radiotherapy. Gamma rays are produced spontaneously as certain elements (such as radium, uranium, and cobalt 60) release radiation as they decay. Each element decays at a specific rate and gives off energy in the form of gamma rays and other particles. X-rays and gamma rays have the same effect on cancer cells.

Another technique for delivering radiation to cancer cells is to place radioactive implants directly in a tumor or body cavity. This is called internal radiotherapy, and brachytherapy, interstitial irradiation, and intracavitary irradiation are types of internal radiotherapy. In this treatment, the radiation dose is concentrated in a small area. Internal radiotherapy is frequently used for cancers of the tongue, uterus, and cervix.

Several new approaches to radiation therapy are being evaluated to determine their effectiveness in treating cancer. One such technique is intraoperative irradiation, in which a large dose of external radiation is directed at the tumor and surrounding tissue during surgery.

Another investigational approach is particle beam radiation therapy. This type of therapy differs from photon radiotherapy in that it involves the use of fast-moving subatomic particles to treat localized cancers. A very sophisticated machine is needed to produce and accelerate the particles required for this procedure. Some particles (neutrons, pions, and heavy ions) deposit more energy along the path they take through tissue than do x-rays or gamma rays, thus causing damage to the cells they hit. This type of radiation is often referred to as high linear energy transfer (high LET) radiation.

Another recent radiotherapy research has focused on the use of radiolabeled antibodies to deliver doses of radiation directly to the cancer site (radioimmunotherapy).

The invention further relates to a method for the treatment of a tumorigenic disease, wherein a therapeutically effective amount of at least one oncolytic virus and at least on antiangiogentic agent is administered to a patient.

All embodiments discussed above with respect to the compositions, substances, combinations and uses of the invention also apply to this method of the invention.

The combination of viral infection with antiangiogenic treatment produces tumor cures which are greater than those produced by either treatment alone. Cell-targeting with oncolytic viruses and inhibitors of angiogenesis to simultaneously suppress tumor growth and metastasis provides a new conceptual basis for increasing the therapeutic ratio in cancer treatment.

The invention is further explained by the following examples and figures, which are not intended to limit the scope of the invention.

### Example 1

Currently a clinical phase I/II study is performed to test safety and efficacy of increasing doses of the oncolytic HSV NV1020 for the treatment of liver metastases in patients suffering from colorectal carcinoma. When entering the clinical trial the patients are progressive despite treatment with chemotherapeutics and/or monoclonal antibodies such as Avastin® or Erbitux®.

4 infusions of NV1020 in 4 dose cohorts (3×10⁶, 1×10⁷, 3×10⁷ and 1×10⁸ pfu) were administered loco-regionally into the hepatic artery to the liver at a weekly schedule followed by follow-on therapy (e.g. chemotherapy and/or an antiangiogenic agent such as Avastin®).

Besides safety data tumors are assessed after the 4th infusion of NV1020 and after the follow-on therapy through whole body CT and PET scans. As serological responses CEA levels and inflammatory cytokines are measured. Time to progression and survival data are collected.

### Example 2

Example 2 describes the treatment of a 63 year-old Caucasian female who presented with poorly differentiated colorectal adenocarcinoma in April 2003. Post resection, adjuvant chemotherapy with 5-FU/Leucovorin was started (May 2003-September 2003). In May 2005, patient was diagnosed with liver metastases and treated with Bevacizumab+FOLFOX (August-September 2005), followed by Capecitabine (Xeloda®)+CPT-11/Irinotecan (Camtosar®) (August-September 2006; FIG. 1).

The patient received 4 weekly intra-arterial infusions of oncolytic NV1020 (1×10⁸ pfu). None of the 4 NV1020-infusions was associated with significant virus-related side effects. Patient received 2 cycles of CPT-11/Irinotecan (Camtosar®) plus Cetuximab (Erbitux®) uneventfully after NV1020 infusion, per protocol.

Subsequently, 3 months follow-up CT scans in December 2006 showed stabilization of disease. PET scans showed a reduced FDG uptake with a 54% decreased SUV value in the liver metastases (FIG. 2).

6 months post treatment (March 2007), CT, FDG-PET and PET-CT scan demonstrated that stabilization of disease was still maintained (FIG. 3). CEA levels dropped from 39.4 ng/ml at the beginning of the study to 13.1 ng/ml 6 months later. KPS score remained at 100% over time of the study.

This case reports shows an unexpectedly lasting radiological benefit to second line treatment using NV1020/CPT11/Cetuximab in a patient with progressive metastatic colorectal cancer.

Regional delivery of NV1020 might have activity alone and appeared to augment efficacy of subsequent CPT-11/Cetuximab treatment. Notably the patient was in a progressive disease state post treatment with a large number anticancer treatments (Bevacizumab, FOLFOX (combination of Oxaliplatin, Folic acid and 5-FU), Capecitabine and CPT-11) when included into the study. Still, the patient showed a marked response to the combination treatment of NV1020/CPT11/Cetuximab.

### SHORT LEGEND TO THE FIGURES

FIG. 1: CT scans of patient at study start: Coronal (1), Sagittal (2), Transversal CT without (3) and with contrast fluid (4) showing intrahepatic lesion

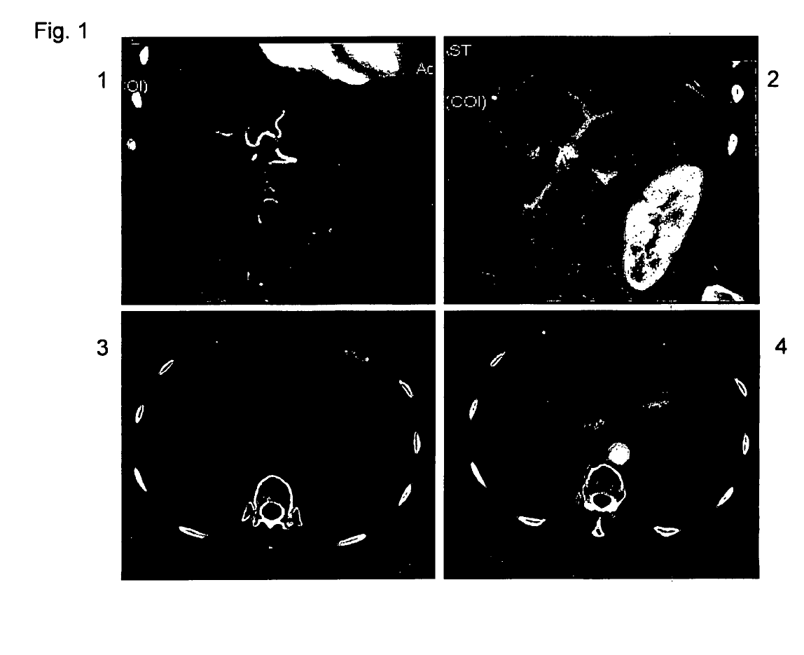

FIG. 2: FDG PET Scan prior to NV1020 (1), vs. post 4×NV1020 and post 2nd line chemotherapy at 3 months (2) and at 6 months (3).

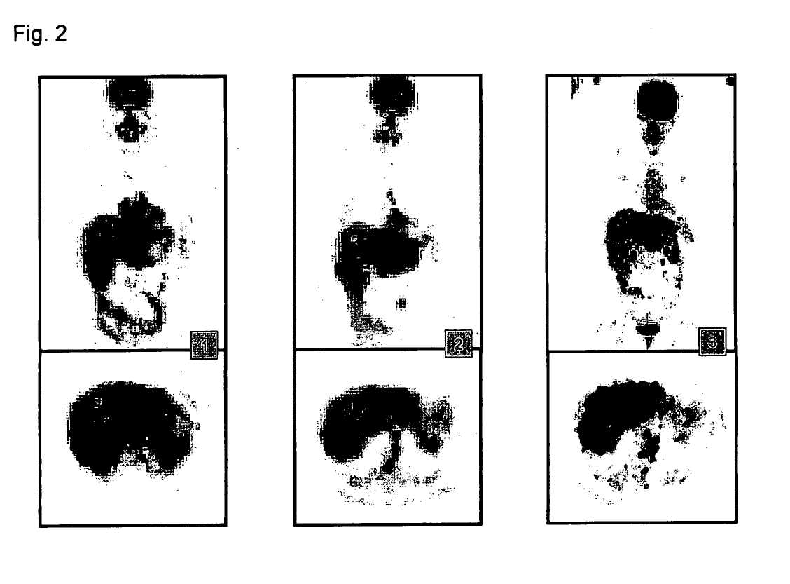

FIG. 3: Liver Metastases at 6 months following 4×NV1020 (1×10⁸ pfu i.a.) and 2nd line chemotherapy with CPT-11+Cetuximab (i.v.): CT (1), PET (2), PET-CT (3)

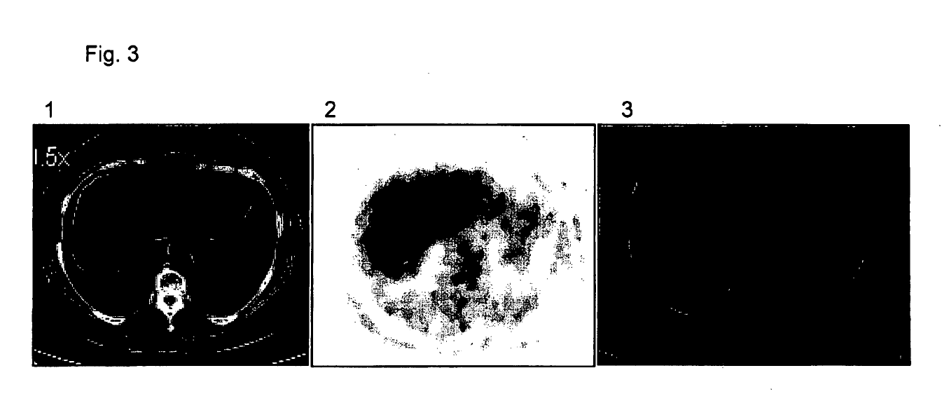

### LITERATURE

Amino N., Ideyama Y., Yamano M., Kuromitsu S., Tajinda K., Samizu K., Hisamichi H., Matsuhisa A., Shirasuna K., Kudoh M., Shibasaki M., 2006. YM-359445, an orally bioavailable vascular endothelial growth factor receptor-2 tyrosine kinase inhibitor, has highly potent antitumor activity against established tumors. Clin. Cancer Res. 12, 1630-1638.
Blood C. H., Zetter B. R., 1990. Tumor interactions with the vasculature: angiogenesis and tumor metastasis. Biochim. Biophys. Acta 1032, 89-118 (review).
Breakefield X. O., Geller A. I., 1987. Gene transfer into the nervous system. Molec. Neurobiol. 1, 339-371 (review).
Brekken R. A., Thorpe P. E., 2001. Vascular endothelial growth factor and vascular targeting of solid tumors. Anticancer Res. 21, 4221-4229 (review).
Cardones A. R., Banez L. L. 2006. VEGF inhibitors in cancer therapy. Curr. Pharm. 12, 387-394 (review).
Cersosimo R. J., Carr D., 1996. Prostate cancer: current and evolving strategies. Am. J. Health Syst. Pharm. 53, 381-396.
Cho J., Kim Y., 2002. Sharks: a potential source of antiangiogenic factors and tumor treatments. Mar. Biotechnol. (NY) 4, 521-525.
Chou, J., Kern E. R., Whitley R. J., Roizman B., 1990. Mapping of herpes simplex virus-1 neurovirulence to gamma 134.5, a gene nonessential for growth in culture. Science 250, 1262.
Ciardiello, F., 2005, Epidermal growth factor receptor inhibitors in cancer treatment. Future Oncol. April; 1(2):221-34. Review.
Coen D. M., Weinheimer S. P., McKnight S. L., 1986. A genetic approach to promoter recognition during trans induction of viral gene expression. Science 234:53-59.
Coen D. M., 1990. Molecular genetics of animal viruses. In: Fields B. N., Knipe D., Chanock R., Hirsch M., Melnick J., Monath T., Roizman B. (editors), Virology, 2nd Ed., New York, Raven Press, 123-150.
Coukos G. et al., 2000. Oncolytic Herpes Simplex Virus-1 Lacking ICP34.5 Induces p53-independent Death and Is Efficacious against Chemotherapy resistant Ovarian Cancer. Clin Cancer Res 6, 3342-53.
Erlichman C., Hidalgo M., Boni J. P., Martins P., Quinn S. E., Zacharchuk C., Amorusi P., Adjei A. A., Rowinsky E. K., 2006. Phase I study of EKB-569, an irreversible inhibitor of the epidermal growth factor receptor, in patients with advanced solid tumors. J. Clin. Oncol., 24(15), 2252-60.
Ferrara N., 2005. VEGF as a therapeutic target in cancer. Oncology. 69 Suppl 3, 11-16 (review)
Folkman J., Haudenschild C., 1980. Angiogenesis in vitro. Nature 288, 551-556.
Fu X., Zhang X., 2002. Potent systemic antitumor activity from an oncolytic herpes simplex virus of syncytial phenotype. Cancer Res. 62, 2306-2312.
Geller A. I., 1988. A new method to propagate defective HSV-1 vectors. Nucl. Acid Res. 16, 5690.
Geller A. I., Breakefield X. O., 1988. A defective HSV-1 vector expresses Escherichia coli beta-galactosidase in cultured peripheral neurons. Science 241, 1667-1669.
Geller A. I., Freese A., 1990. Infection of cultured central nervous system neurons with a defective herpes simplex virus 1 vector results in stable expression of Escherichia coli beta-galactosidase. Proc. Natl. Acad. Sci. U.S.A. 87, 1149-1153.
Goldberg R. M., 2005. Advances in the treatment of metastatic colorectal cancer. Oncologist 10, 40-48 (review).
Goss, 1978. The Physiology of Growth. Academic Press, New York, 120-137
Graff J. R., McNulty A. M., Hanna K. R., Konicek B. W., Lynch R. L., Bailey S. N., Banks C., Capen A., Goode R., Lewis J. E., Sams L., Huss K. L., Campbell R. M., Iversen P. W., Neubauer B. L., Brown T. J., Musib L., Geeganage S., Thornton D., 2005. The protein kinase Cbeta-selective inhibitor, Enzastaurin (LY317615.HCl), suppresses signaling through the AKT pathway, induces apoptosis, and suppresses growth of human colon cancer and glioblastoma xenografts. Cancer Res. 65, 7462-7469.
Grant S, Qiao L, Dent P. Roles of ERBB family receptor tyrosine kinases, and downstream signaling pathways, in the control of cell growth and survival. Front Biosci. 2002 February 1;7:d376-89. Review.
Guedez L, Rivera A M, Salloum R, Miller M L, Diegmueller J J, Bungay P M, Stetler-Stevenson W G, 2003. Quantitative assessment of angiogenic responses by the directed in vivo angiogenesis assay. Am J Pathol 162(5), 1431-9.
Heath E. I., Grochow L. B., 2000. Clinical potential of matrix metalloprotease inhibitors in cancer therapy. Drugs 59, 1043-1055 (review).
Hess-Stumpp H., Haberey M., Thierauch K. H., 2005. PTK 787/ZK 222584, a tyrosine kinase inhibitor of all known VEGF receptors, represses tumor growth with high efficacy. Chembiochem. 6, 550-557.
Heymach J. V., Nilsson M., Blumenschein G., Papadimitrakopoulou V., Herbst R., 2006. Epidermal growth factor receptor inhibitors in development for the treatment of non-small cell lung cancer. Clin Cancer Res. 12(14 Pt 2), 4441s-4445s (review).
Isayeva T., Kumar S., Ponnazhagan S., 2004. Anti-angiogenic gene therapy for cancer. Int. J. Oncol. 25, 335-343 (review).
Jain R. K., Duda D. G., Clark J. W., Loeffler J. S., 2006. Lessons from phase III clinical trials on anti-VEGF therapy for cancer. Nat. Clin. Pract. Oncol. 3, 24-40.
Jayson G. C., Mulatero C., Ranson M., Zweit J., Jackson A., Broughton L., Wagstaff J., Hakansson L., Groenewegen G., Lawrance J., Tang M., Wauk L., Levitt D., Marreaud S., Lehmann F. F., Herold M., Zwierzina H.; European Organisation for Research and Treatment of Cancer (EORTC), 2005. Phase I investigation of recombinant anti-human vascular endothelial growth factor antibody in patients with advanced cancer. Eur. J. Cancer 41, 555-563.
Khalil M. Y., Grandis J. R., Shin D. M., 2003. Targeting epidermal growth factor receptor: novel therapeutics in the management of cancer. Expert Rev. Anticancer Ther., 3, 367-80 (review).
Kamiyama H., Zhou G., Roizman B. (,2006. Herpes simplex virus 1 recombinant virions exhibiting the amino terminal fragment of urokinase-type plasminogen activator can enter cells via the cognate receptor. Gene Ther. 13, 621-629.
Kim K. J., Li B., Winer J., Armanini M., Gillett N., Phillips H. S., Ferrara N., 1993. Inhibition of vascular endothelial growth factor-induced angiogenesis suppresses tumour growth in vivo. Nature 362, 841-844.
Kim D. H., 2000. Replication-selective microbiological agents: fighting cancer with targeted germ warfare. J. Clin. Invest. 105, 837-839.
Klagsbrun M., D'Amore P. A., 1991. Regulators of angiogenesis. Annu. Rev. Physiol. 53, 217-239 (review).
Kuenen B. C., Giaccone G., Ruijter R., Kok A., Schalkwijk C., Hoekman K., Pinedo H. M., 2005. Dose-finding study of the multitargeted tyrosine kinase inhibitor SU6668 in patients with advanced malignancies. Clin. Cancer Res. 11, 6240-6246.
Kramm C. M., Chase M., Herrlinger U., Jacobs A., Pechan P. A., Rainov N. G., Sena-Esteves M., Aghi M., Barnett F. H., Chiocca E. A., Breakefield X. O., 1997. Therapeutic efficiency and safety of a second-generation replication-conditional HSV1 vector for brain tumor gene therapy. Hum. Gene Ther., 8: 2057-2068.
Kunstfeld R., Wickenhauser G., Michaelis U., Teifel M., Umek W., Naujoks K., Wolff K., Petzelbauer P., 2003. Paclitaxel encapsulated in cationic liposomes diminishes tumor angiogenesis and melanoma growth in a “humanized” SCID mouse model. J. Invest. Dermatol. 120, 476-482.
Lenz H. J., 2005. Antiangiogenic agents in cancer therapy. Oncology 19, 17-25 (review).
Liu X. Y., Gu J. F., Shi W. F., 2005. Targeting gene-virotherapy for cancer. Acta Biochim. Biophys. Sin(Shanghai) 37, 581-587 (review).
Martuza R. L., 2000. Conditionally replicating herpes vectors for cancer therapy. J. Clin. Invest. 105, 841-846 (review).
Matz B., Subak-Sharpe J. H., Preston V. G., 1983. Physical mapping of temperature-sensitive mutations of herpes simplex virus type 1 using cloned restriction endonuclease fragments. J. Gen. Virol. 64, 2261-2270.
Matzku S., Zoller M., 2001. Specific immunotherapy of cancer in elderly patients. Drugs Aging. 18, 639-664 (review).
Meignier B., Longnecker R., Roizman B., 1988. In vivo behavior of genetically engineered herpes simplex viruses R7017 and R7020: construction and evaluation in rodents. J. Infect. Dis. 158: 602-614.
Menotti L., Cerretani A., Campadelli-Fiume G., 2006. A Herpes Simplex Virus Recombinant That Exhibits a Single-Chain Antibody to HER2/neu Enters Cells through the Mammary Tumor Receptor, Independently of the gD Receptors. J. Virol. 80, 5531-5539.
Mineta T., Rabkin S. D., Yazaki T., Hunter W. D., Martuza R. L., 1995. Attenuated multi-mutated herpes simplex virus-1 for the treatment of malignant gliomas. Nature Medicine 1, 938-943.
Mocarski E. S., Post L. E., Roizman B., 1980. Molecular engineering of the herpes simplex virus genome: insertion of a second L-S junction into the genome causes additional genome inversions. Cell 22, 243-255.
Morgensztern D., Govindan R., 2006. Clinical trials of antiangiogenic therapy in non-small cell lung cancer: focus on bevacizumab and ZD6474. Expert Rev Anticancer Ther. 6, 545-551.
Motzer R. J., Hoosen S., Bello C. L., Christensen J. G., 2006. Sunitinib malate for the treatment of solid tumours: a review of current clinical data. Expert Opin Investig Drugs 15, 553-561.
Murakami H., Handa H., 2006. [New treatment strategy of multiple myeloma for cure] Gan To Kagaku Ryoho 33, 417-423 (review, Japanese).
Nakamura H., Mullen J. T., Chandrasekhar S., Pawlik T. M., Yoon S. S., Tanabe K. K., 2001. Multimodality therapy with a replication-conditional herpes simplex virus 1 mutant that expresses yeast cytosine deaminase for intratumoral conversion of 5-fluorocytosine to 5-fluorouracil. Cancer Res. 61, 5447-5452.
Nakamura H., Kasuya H., Mullen J. T., Yoon S. S., Pawlik T. M., Chandrasekhar S., Donahue J. M., Chiocca E. A., Chung R. Y., Tanabe K. K., 2002. Regulation of herpes simplex virus gamma(1)34.5 expression and oncolysis of diffuse liver metastases by Myb34.5. J. Clin. Invest. 109, 871-882.
Nam J O, Kim J E, Jeong H W, Lee S J, Lee B H, Choi J Y, Park R W, Park J Y, Kim I S., 2003. Identification of the α_{ν}β₃ integrin-interacting motif of βig-h3 and its anti-angiogenic effect. J Biol Chem 278, 28, 25902-909.
[No authors listed], 2004. AE 941. Drugs R D 5, 83-89 (review).
[No authors listed], 2002. ECM625 Datasheet/Insert Revision B: 41075, CHEMICON.
[No authors listed], 2004, DIVAA Cultrex Instructions for Use, Trevigen, Inc. Gaithersburg Md.
O'Donnell A., Padhani A., Hayes C., Kakkar A. J., Leach M., Trigo J. M., Scurr M., Raynaud F., Phillips S., Aherne W., Hardcastle A., Workman P., Hannah A., Judson I., 2005. A Phase I study of the angiogenesis inhibitor SU5416 (semaxanib) in solid tumours, incorporating dynamic contrast MR pharmacodynamic end points. Br. J. Cancer. 93, 876-883.
Palella T. D., Silverman L. J., Schroll C. T., Homa F. L., Levine M., Kelley W. N., 1988. Herpes simplex virus-mediated human hypoxanthine-guanine phosphoribosyl-transferase gene transfer into neuronal cells. Molec. Cell. Biol. 8, 457-460.
Pawlik T. M., Nakamura H., Yoon S. S., Mullen J. T., Chandrasekhar S., Chiocca E. A., Tanabe K. K., 2000. Oncolysis of diffuse hepatocellular carcinoma by intravascular administration of a replication-competent, genetically engineered herpesvirus. Cancer Res. 60, 2790-2795.
Post L. E., Roizman B., 1981. A generalized technique for deletion of specific genes in large genomes: alpha gene 22 of herpes simplex virus 1 is not essential for growth. Cell 25, 227-232.
Post D. E., Fulci G., Chiocca E. A., Van Meir E. G., 2004. Replicative oncolytic herpes simplex viruses in combination cancer therapies. Curr. Gene Ther. 4, 41-51 (review).
Ramnath N., Creaven P. J., 2004. Matrix metalloproteinase inhibitors. Curr. Oncol. Rep. 6, 96-102 (review).
Rosenberg S. A., 1985. Combined modality therapy of cancer. What is it and when does it work? New Engl. J. Med. 312, 1512-1514.
Saetern A. M., Flaten G. E., Brandi M., 2004. A method to determine the incorporation capacity of camptothecin in liposomes. AAPS Pharm. Sci. Tech. 5, e40.
Sambrook J., Fritsch E. F., Maniatis T., 1989. Molecular Cloning: A Laboratory Manual. Cold Spring Harbor Laboratory Press, New York, 2nd Ed.
Sequist L. V., 2007. Second-generation epidermal growth factor receptor tyrosine kinase inhibitors in non-small cell lung cancer. The Oncologist 12, 325-330.
Shih M.-F. et al., 1985. Herpes Simplex Virus as a Vector for Eukaryotic Viral Genes in Lerner, R. A. et al., eds., Vaccines 85, Cold Spring Harbor Press, Cold Spring Harbor, N.Y., 177-180.
Shimamura T., Ji H., Minami Y., Thomas R. K., Lowell A. M., Shah K., Greulich H., Glatt K. A., Meyerson M., Shapiro G. I., Wong K.K., 2006. Non-small-cell lung cancer and Ba/F3 transformed cells harboring the ERBB2 G776insV_G/C mutation are sensitive to the dual-specific epidermal growth factor receptor and ERBB2 inhibitor HKI-272. Cancer Res. 66(13), 6487-91.
Smiley J. R., 1980. Construction in vitro and rescue of a thymidine kinase-deficient deletion mutation of herpes simplex virus. Nature 285, 333-335.
Speidel C. C., 1933. Studies of living nerves. II. Activities of amoeboid growth cones, sheath cells, and myelin segments, as revealed by prolonged observation of individual nerve fibers in frog tadpoles. Anat. 52, 1-79.
Strieth S., Eichhorn M. E., Sauer B., Schulze B., Teifel M., Michaelis U., Dellian M., 2004. Neovascular targeting chemotherapy: encapsulation of paclitaxel in cationic liposomes impairs functional tumor microvasculature. Int. J. Cancer 110, 117-124.
Strieth S., Eichhorn M. E., Sutter A., Jonczyk A., Berghaus A., Dellian M., 2006. Antiangiogenic combination tumor therapy blocking alpha(ν)-integrins and VEGF-receptor-2 increases therapeutic effects in vivo. Int. J. Cancer 119, 423-431.
Teo S. K., 2005. Properties of thalidomide and its analogues: implications for anticancer therapy. AAPS J. 7, E14-E19 (review).
Thorpe P. E., 2004. Vascular targeting agents as cancer therapeutics. Clin. Cancer Res. 10, 415-427 (review).
Thurston G., McLean J. W., Rizen M., Baluk P., Haskell A., Murphy T. J., Hanahan D., McDonald D. M., 1998. Cationic liposomes target angiogenic endothelial cells in tumors and chronic inflammation in mice. J. Clin. Invest. 101, 1401-1413.
Todo T., Martuza R. L., Rabkin S. D., Johnson P. A., 2001. Oncolytic herpes simplex virus vector with enhanced MHC class I presentation and tumor cell killing. Proc. Natl. Acad. Sci. U.S.A. 98, 6396-6401.
Traxler P., Allegrini P. R., Brandt R., Brueggen J., Cozens R., Fabbro D., Grosios K., Lane H. A., McSheehy P., Mestan J., Meyer T., Tang C., Wartmann M., Wood J., Caravatti G., 2004. AEE788: a dual family epidermal growth factor receptor/ErbB2 and vascular endothelial growth factor receptor tyrosine kinase inhibitor with antitumor and antiangiogenic activity. Cancer Res. 64, 4931-4941.
Umeda N., Kachi S., Akiyama H., Zahn G., Vossmeyer D., Stragies R., Campochiaro P., 2006. Suppression and Regression of Choroidal Neovascularization by Systemic Administration of an {alpha}5{beta}1 Integrin Antagonist. Mol. Pharmacol. 9; [Epub ahead of print]
Varghese, S. et al., 2006. Systemic oncolytic herpes virus therapy of poorly immunogenic prostate cancer metastatic to lung. Clin Cancer Res 12(9), 2919-27.
Wakelee H. A., Schiller J. H., 2005. Targeting angiogenesis with vascular endothelial growth factor receptor small-molecule inhibitors: novel agents with potential in lung cancer. Clin. Lung Cancer 7, 31-38 (review).
Wedge S. R., Ogilvie D. J., Dukes M., Kendrew J., Curwen J. O., Hennequin L. F., Thomas A. P., Stokes E. S., Curry B., Richmond G. H., Wadsworth P. F., 2000. ZD4190: an orally active inhibitor of vascular endothelial growth factor signaling with broad-spectrum antitumor efficacy. Cancer Res. 60, 970-975.
Wilkinson-Berka J. L., Jones D., Taylor G., Jaworski K., Kelly D. J., Ludbrook S. B., Willette R. N., Kumar S., Gilbert R. E., 2006. SB-267268, a nonpeptidic antagonist of alpha(ν)beta3 and alpha(ν)beta5 integrins, reduces angiogenesis and VEGF expression in a mouse model of retinopathy of prematurity. Invest. Opthalmol. Vis. Sci. 47, 1600-1605.
Wittekind C., Neid M., 2005. Cancer invasion and metastasis. Oncology 69 Suppl 1, 14-16 (review).
Zakarija A., Soff G., 2005. Update on angiogenesis inhibitors. Curr Opin Oncol. 17, 578-583 (review).
Zhang W., Ran S., Sambade M., Huang X., Thorpe P. E., 2002. A monoclonal antibody that blocks VEGF binding to VEGFR2 (KDR/Flk-1) inhibits vascular expression of Flk-1 and tumor growth in an orthotopic human breast cancer model. Angiogenesis 5, 35-44.
Zhong H. and Bowen J. P, 2007. Molecular design and clinical development of VEGFR kinase inhibitors. Curr. Top. Med. Chem. 7, 1379-93.
Zhou G., Roizman B., 2006. Construction and properties of a herpes simplex virus 1 designed to enter cells solely via the IL-13 alpha2 receptor. Proc. Natl. Acad. Sci. U.S.A. 103, 5508-5513.
Zhu Z., 2007. Targeted cancer therapies based on antibodies directed against epidermal growth factor receptor: status and perspectives. Acta Pharmacologica Sinicia 28(9), 1476-93.

1-71. (canceled)

72. A combination of at least one oncolytic virus and at least one antiangiogenic agent.

73. The combination of claim 72, wherein said oncolytic virus is selected from the group consisting of herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SIN), and sendai virus (SV).

74. The combination of claim 73, wherein said oncolytic virus is an attenuated herpes virus, in particular wherein the herpes virus is herpes simplex virus 1 (HSV-1), more in particular wherein said attenuated HSV-1 is rendered incapable of expressing an active gene product by nucleotide insertion, deletion, substitution, inversion, and/or duplication.

75. The combination of claim 74, wherein said attenuated HSV-1 has a deletion of an inverted repeat region of the HSV genome such that the region is rendered incapable of expressing an active gene product from one copy only of each of α0, α4, ORFO, ORFP, and γ₁34.5, especially wherein said attenuated HSV-1 is NV1020, or wherein said attenuated HSV-1 is rendered incapable of expressing an active gene product from both copies of γ₁34.5.

76. The combination of claim 75, wherein said oncolytic virus is further attenuated by an attenuating mutation of one or more genes selected from the group consisting of γ₁34.5, U_{L}2, U_{L}3, U_{L}4, U_{L}10, U_{L}11, U_{L}12, U_{L}12.5, U_{L}13, U_{L}16, U_{L}20, U_{L}21, U_{L}23, U_{L}24, U_{L}39, U_{L}40, U_{L}41, U_{L}43, U_{L}43.5, U_{L}44, U_{L}45, U_{L}46, U_{L}47, U_{L}50, U_{L}51, U_{L}53, U_{L}55, U_{L}56, α22, U_{S}1.5, U_{S}2, U_{S}3, U_{S}4, U_{S}5, U_{S}7, U_{S}8, U_{S}8.5, U_{S}9, U_{S}10, U_{S}11, α47, Ori_{S}TU, and LATU, preferably U_{L}39, U_{L}56, and α47, especially the attenuated HSV-1 is G207 or G47Δ.

77. The combination of claim 72, wherein the herpes simplex virus further contains foreign DNA.

78. The combination of claim 72, wherein said antiangiogenic agent is selected from the group consisting of agents that target the vascular endothelial growth factor (VEGF) pathway, an integrin, a matrix metalloproteinase (MMP) and/or protein kinase C beta (PKCβ), or a combination thereof.

79. The combination of claim 78, wherein
a) said antiangiogenic agents targeting MMPs or integrins are chimeric, humanized, or fully human monoclonal antibodies, or
b) said antiangiogenic agents targeting a MMP is selected from the group consisting of marimastat, metastat (COL 3), BAY-129566, CGS-27023A, prinomastat (AG-3340), and BMS-275291, or
c) said antiangiogenic agents targeting an integrin is selected from the group consisting of SB-267268, JSM6427, and EMD270179, or
d) said VEGF pathway targeting agent is:
i) an antibody or a fragment thereof against a member of the VEGF family (VEGF, placental growth factor (P1GF), VEGF-B, VEGF-C, VEGF-D) or their receptors (VEGFR-1, -2, -3), in particular wherein said antibody is a monoclonal antibody, more in particular wherein said monoclonal antibody is Bevacizumab (Avastin®), 2C3, or HuMV833 or a combination thereof, and/or
ii) a small molecule tyrosine kinase inhibitor of VEGF receptors, and/or
iii) a soluble VEGF receptor, and/or
iv) a ribozyme which specifically targets VEGF mRNA, or
e) said PKCβ-selective inhibitor is Enzastaurin (LY317615).

80. The combination of claim 79, wherein said tyrosine kinase inhibitor is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, and YM-359445, preferably wherein the tyrosine kinase inhibitor is ZD6474, or wherein said soluble VEGF receptor is VEGF-Trap, or wherein said ribozyme specifically targeting VEGF mRNA is Angiozyme™.

81. The combination of claim 72, wherein said antiangiogenic agent is selected from the group consisting of a cationic liposome, a Vascular Targeting Agent (VTA), Neovastat (AE-941), U-995, Squalamine, and Thalidomide or one of its immunomodulatory analogs, or a combination thereof, in particular wherein said immunomodulatory analog of Thalidomide is selected from the group consisting of lenalidomide, Revlimid, CC-5013, CC-4047, and ACTIMID, or wherein said VTA is a small molecule or a ligand-based agent, in particular wherein said small molecule VTA is selected from the group consisting of combretastatin A-4 disodium phosphate (CA4P), ZD6126, AVE8062, Oxi 4503, DMXAA and TZT1027, preferably the small molecule agent is CA4P, or wherein said ligand-based VTA uses an antibody, peptide or growth factor.

82. The combination of claim 81, wherein said cationic liposome carries an antimitotic agent, in particular wherein said antimitotic agent is Na-Camptothecin or a taxane, preferably paclitaxel or a derivative thereof, or wherein said cationic liposomal preparation comprises at least one cationic lipid and at least one neutral and/or anionic lipid and said cationic liposomal carries an antimitotic agent, in particular wherein said cationic liposomal preparation comprises 1,2-dioleoyl-3-trimethylammonium propane (DOTAP) and 1,2-dioleoyl-sn-glycero-3-phosphocholine (DOPC).

83. The combination of claim 72, wherein said antiangiogenic agent is a receptor antagonist of epidermal growth factor receptor (EGFR) signaling pathway, in particular wherein said receptor antagonist of epidermal growth factor receptor (EGFR) is an EGFR tyrosine kinase inhibitor, more in particular wherein said EGFR tyrosine kinase inhibitor is an anti-EGFR monoclonal antibody, and most in particular wherein said monoclonal antibody is cetuximab (Erbitux®), panitumumab (Vectibix®), nimotuzumab, matuzumab, zalutuzumab, mAb 806, or IMC-11F8.

84. The combination of claim 72, wherein said antiangiogenic agent is a tyrosine kinase inhibitor, in particular wherein said tyrosine kinase inhibitor is selected from the group consisting of agents that target the vascular endothelial growth factor receptor (VEGFR) pathway, the epidermal growth factor receptor (EGFR) pathway, the platelet-derived growth factor receptor (P1GFR), the fibroblast growth factor receptor (FGFR), and ErbB2 or an agent that targets a combination thereof, or wherein said tyrosine kinase inhibitor is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, gefitinib (Iressa®), erlotinib (Tarceva®), EKB-569, HKI-272, and Cl-1033, preferably wherein the tyrosine kinase inhibitor is ZD6474, or wherein said tyrosine kinase inhibitor is a monoclonal antibody, in particular wherein said monoclonal antibody is Bevacizumab (Avastin®), 2C3, HuMV833, cetuximab (Erbitux®), panitumumab (Vectibix®), nimotuzumab, matuzumab, zalutuzumab, mAb 806, or IMC-11F8.

85. A combination of at least one oncolytic virus and at least one tyrosine kinase inhibitor.

86. The combination of claim 85, wherein (i) said tyrosine kinase inhibitor
a) is selected from the group consisting of agents that target the vascular endothelial growth factor receptor (VEGFR) pathway, the epidermal growth factor receptor (EGFR) pathway, the platelet-derived growth factor receptor (P1GFR), the fibroblast growth factor receptor (FGFR), and ErbB2 or an agent that targets a combination thereof, or
b) targets the vascular endothelial growth factor receptor (VEGFR) and is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, Bevacizumab (Avastin®), 2C3, and HuMV833, preferably wherein the tyrosine kinase inhibitor is ZD6474, or
c) targets the epidermal growth factor receptor (EGFR) and is selected from the group consisting of AEE788, ZD6474, gefitinib (Iressa®), erlotinib (Tarceva®), EKB-569, HKI-272, CI-1033, cetuximab (Erbitux®), panitumumab (Vectibix®), nimotuzumab, matuzumab, zalutuzumab, mAb 806, and IMC-11F8, or
d) targets the platelet-derived growth factor receptor (P1GFR), the fibroblast growth factor receptor (FGFR), ErbB2 or a combination of said receptors, and is selected from the group consisting of SU6668, vatalanib (PTK787/ZK222584) and AEE788, or
e) is a monoclonal antibody, in particular wherein said monoclonal antibody is Bevacizumab (Avastin®), 2C3, HuMV833, cetuximab (Erbitux®), panitumumab (Vectibix®), nimotuzumab, matuzumab, zalutuzumab, mAb 806, or IMC-11F8, or
wherein (ii) said oncolytic virus is selected from the group consisting of herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SIN), and sendai virus (SV).

87. A method for the treatment of a tumorigenic disease, wherein
a) at least one oncolytic virus is administered simultaneously, sequentially or separately in combination with at least one antiangiogenic agent, at least one receptor antagonist of epidermal growth factor receptor (EGFR) signaling pathway or at least one tyrosine kinase inhibitor, or
b) at least one antiangiogenic agent, at least one receptor antagonist of epidermal growth factor receptor (EGFR) signaling pathway or at least one tyrosine kinase inhibitor is administered simultaneously, sequentially or separately in combination with at least one oncolytic virus.

88. The method of treatment of claim 87, wherein
a) the oncolytic virus is selected from the group consisting of herpes viruses, Adenovirus, Adeno-associated virus, influenza virus, reovirus, vesicular stomatitis virus (VSV), Newcastle virus, vaccinia virus, poliovirus, measles virus, mumps virus, sindbis virus (SIN), and sendai virus (SV), or
b) the antiangiogenic agent is selected from the group consisting of agents that target the vascular endothelial growth factor (VEGF) pathway, an integrin, a matrix metalloproteinase (MMP) and/or protein kinase C beta (PKCβ), or a combination thereof, or
c) the receptor antagonist of epidermal growth factor receptor (EGFR) signaling pathway is an EGFR tyrosine kinase inhibitor, in particular wherein said EGFR tyrosine kinase inhibitor is an anti-EGFR monoclonal antibody, more in particular wherein said monoclonal antibody is cetuximab (Erbitux®), panitumumab (Vectibix®), nimotuzumab, matuzumab, zalutuzumab, mAb 806, or IMC-11F8, or
d) the tyrosine kinase inhibitor
i) is selected from the group consisting of agents that target the vascular endothelial growth factor receptor (VEGFR) pathway, the epidermal growth factor receptor (EGFR) pathway, the platelet-derived growth factor receptor (P1GFR), the fibroblast growth factor receptor (FGFR), ErbB2 or an agent that targets a combination thereof, or
ii) targets the vascular endothelial growth factor receptor (VEGFR) and is selected from the group consisting of sunitinib (SU11248; Sutent®), SU5416, SU6668, vatalanib (PTK787/ZK222584), AEE788, ZD6474, ZD4190, AZD2171, GW786034, sorafenib (BAY 43-9006), CP-547,632, AG013736, YM-359445, Bevacizumab (Avastin®), 2C3, and HuMV833, preferably wherein the tyrosine kinase inhibitor is ZD6474, or
iii) targets the epidermal growth factor receptor (EGFR) and is selected from the group consisting of AEE788, ZD6474, gefitinib (Iressa®), erlotinib (Tarceva®), EKB-569, HKI-272, Cl-1033, cetuximab (Erbitux®), panitumumab (Vectibix®), nimotuzumab, matuzumab, zalutuzumab, mAb 806, and IMC-11F8, or
iv) targets the platelet-derived growth factor receptor (P1GFR), the fibroblast growth factor receptor (FGFR), ErbB2 or a combination of said receptors, and is selected from the group consisting of SU6668, vatalanib (PTK787/ZK222584), and AEE788, or
v) is a monoclonal antibody, in particular wherein said monoclonal antibody is Bevacizumab (Avastin®), 2C3, HuMV833, cetuximab (Erbitux®), panitumumab (Vectibix®), nimotuzumab, matuzumab, zalutuzumab, mAb 806, or IMC-11F8, or
e) the tumor is
i) contacted first with the virus and then with the antiangiogenic agent, the receptor antagonist of epidermal growth factor receptor (EGFR) signaling pathway or the tyrosine kinase inhibitor, or
ii) contacted first with the antiangiogenic agent, the receptor antagonist of epidermal growth factor receptor (EGFR) signaling pathway or the tyrosine kinase inhibitor and then with the virus, or
f) said virus is to be administered to the patient by means of local, local-regional or systemic injection of from about 10⁸ to 10¹¹ plaque-forming units, preferably of from about 10⁸ to 10⁹ plaque-forming units, or
g) said treatment is combined with chemotherapy and/or radiotherapy, in particular wherein
aa) said further active chemotherapeutic agent is selected from the group consisting of
(i) an alkylating agent including busulfan, carmustine, chlorambucil, cyclophosphamide (i.e., cytoxan), dacarbazine, ifosfamide, lomustine, mecholarethamine, melphalan, platinum containing compounds like cisplatin and carboplatin, procarbazine, streptozocin, and thiotepa, preferably platinum containing compounds like cisplatin and carboplatin.
(ii) an antineoplastic agent including antimitotic agents like paclitaxel or a derivative thereof, bleomycin, dactinomycin, daunorubicin, doxorubicin, idarubicin, mitomycin (e.g., mitomycin C), mitoxantrone, pentostatin, and plicamycin, preferably antimitotic agents like paclitaxel or a derivative thereof,
(iii) an RNA/DNA antimetabolite including fluorodeoxyuridine, capecitabine, cladribine, cytarabine, floxuridine, fludarabine, fluorouracil. gemcitabine, hydroxyurea, mercaptopurine, methotrexate, and thioguanine, preferably 5-fluorouracil (5FU) or capecitabine,
(iv) a natural source derivative including docetaxel, etoposide, irinotecan, paclitaxel, teniposide, topotecan, vinblastine, vincristine, vinorelbine, taxol, prednisone, and tamoxifen, and
(v) an additional chemotherapeutic agent including asparaginase, mitotane, leucovorin, oxaliplatin, DNA topoisomerase inhibiting agents like camptothecin, and anthracyclines like doxorubicin, more in particular wherein the chemotherapeutic agent comprises oxaliplatin and/or irinotecan, optionally wherein the chemotherapeutic agent is FOLFOX (5-fluoruracil, leucovorin and oxaliplatin) or FOLFIRI (5-fluoruracil, leucovorin and irinotecan), or
bb) said radiation therapy uses photon radiation (electromagnetic energy) like X-rays and gamma rays (including the gamma-knife), internal radiotherapy, intraoperative irradiation, particle beam radiation therapy, and radioimmunotherapy.

89. The method of treatment of claim 87, wherein said tumorigenic disease is selected from the group consisting of astrocytoma, oligodendroglioma, meningioma, neurofibroma, glioblastoma, ependymoma, Schwannoma, neurofibrosarcoma, neuroblastoma, pituitary adenoma, medulloblastoma, head and neck cancer, melanoma, prostate carcinoma, renal cell carcinoma, pancreatic cancer, breast cancer, lung cancer, colon cancer, gastric cancer, bladder cancer, liver cancer, bone cancer, rectal cancer, ovarian cancer, sarcoma, gastric cancer, esophageal cancer, cervical cancer, fibrosarcoma, squamous cell carcinoma, neurectodermal, thyroid tumor, Hodgkin's lymphoma, non-Hodgkin's lymphoma, hepatoma, mesothelioma, epidermoid carcinoma, and tumorigenic diseases of the blood, preferably wherein said tumorigenic disease is glioblastoma.

90. The method of treatment of claim 87, wherein said treatment involves the treatment of metastasis of said tumorigenic disease, preferably liver metastasis from colorectal cancer.
